SEQUENCE LISTING (31 sequences)

SEQ ID NO: 1 - protein, 93 aa, Homo sapiens
MGTSLTDEELVTMSVRELNQHLRGLSKEEIIQLKQRRRTLKNRGYAASCRVKRVTQKEELEKQKAELQQEVEKLASENASMKLELDALRSKYE

SEQ ID NO: 2 - protein, 93 aa, Homo sapiens
HIPFPVEKIINLPVVDFNEMMSKEQFNEAQLALIRDIRRRGKNKVAAQNCRKRKLENIVELEQDLDHLKDEKEKLLKEKGENDKSLHLLKKQL

SEQ ID NO: 3 - protein, 16 aa, synthetic construct
IVELEQDLDHLKDEKE

SEQ ID NO: 4 - protein, 16 aa, synthetic construct
DLDHLKDEKEKLLKEK

SEQ ID NO: 5 - protein, 16 aa, synthetic construct
DEKEKLLKEKGENDKS

SEQ ID NO: 6 - protein, 16 aa, synthetic construct
  VARIANT 6: note = X is any unmodified amino acid and a site of attachment of an internal staple of the peptide
  VARIANT 10: note = X is any unmodified amino acid and a site of attachment of an internal staple of the peptide
IVELEXDLDXLKDEKE

SEQ ID NO: 7 - protein, 16 aa, synthetic construct
  VARIANT 6: note = X is any unmodified amino acid and a site of attachment of an internal staple of the peptide
  VARIANT 10: note = X is any unmodified amino acid and a site of attachment of an internal staple of the peptide
DLDHLXDEKXKLLKEK

SEQ ID NO: 8 - protein, 16 aa, synthetic construct
  VARIANT 7: note = X is any unmodified amino acid and a site of attachment of an internal staple of the peptide
  VARIANT 11: note = X is any unmodified amino acid and a site of attachment of an internal staple of the peptide
DEKEKLXKEKXENDKS

SEQ ID NO: 9 - protein, 16 aa, synthetic construct
  VARIANT 5: note = X is any unmodified amino acid and a site of attachment of an internal staple of the peptide
  VARIANT 9: note = X is any unmodified amino acid and a site of attachment of an internal staple of the peptide
IVELXQDLXHLKDEKE

SEQ ID NO: 10 - protein, 16 aa, synthetic construct
  VARIANT 8: note = X is any unmodified amino acid and a site of attachment of an internal staple of the peptide
  VARIANT 4: note = X is any unmodified amino acid and a site of attachment of an internal staple of the peptide
IVEXEQDXDHLKDEKE

SEQ ID NO: 11 - protein, 16 aa, synthetic construct
  VARIANT 3: note = X is any unmodified amino acid and a site of attachment of an internal staple of the peptide
  VARIANT 7: note = X is any unmodified amino acid and a site of attachment of an internal staple of the peptide
IVXLEQXLDHLKDEKE

SEQ ID NO: 12 - protein, 16 aa, synthetic construct
  VARIANT 7: note = X is any unmodified amino acid and a site of attachment of an internal staple of the peptide
  VARIANT 11: note = X is any unmodified amino acid and a site of attachment of an internal staple of the peptide
IVELEQXLDHXKDEKE

SEQ ID NO: 13 - protein, 16 aa, synthetic construct
  VARIANT 8: note = X is any unmodified amino acid and a site of attachment of an internal staple of the peptide
  VARIANT 12: note = X is any unmodified amino acid and a site of attachment of an internal staple of the peptide
EVELEQDXDHLXDEKE

SEQ ID NO: 14 - protein, 16 aa, synthetic construct
  VARIANT 9: note = X is any unmodified amino acid and a site of attachment of an internal staple of the peptide
  VARIANT 13: note = X is any unmodified amino acid and a site of attachment of an internal staple of the peptide
IVELEQDLXHLKXEKE

SEQ ID NO: 15 - protein, 16 aa, synthetic construct
  VARIANT 10: note = X is any unmodified amino acid and a site of attachment of an internal staple of the peptide
  VARIANT 14: note = X is any unmodified amino acid and a site of attachment of an internal staple of the peptide
IVELEQDLDXLKDXKE

SEQ ID NO: 16 - protein, 16 aa, synthetic construct
IVELEXDLDHLKXEKE

SEQ ID NO: 17 - protein, 16 aa, unidentified
  VARIANT 5: note = X is any unmodified amino acid and a site of attachment of an internal staple of the peptide
  VARIANT 9: note = X is any unmodified amino acid and a site of attachment of an internal staple of the peptide
DLDHXKDEXEKLLKEK

SEQ ID NO: 18 - protein, 16 aa, synthetic construct
  VARIANT 4: note = X is any unmodified amino acid and a site of attachment of an internal staple of the peptide
  VARIANT 8: note = X is any unmodified amino acid and a site of attachment of an internal staple of the peptide
DLDXLKDXKEKLLKEK

SEQ ID NO: 19 - protein, 16 aa, synthetic construct
  VARIANT 3: note = X is any unmodified amino acid and a site of attachment of an internal staple of the peptide
  VARIANT 7: note = X is any unmodified amino acid and a site of attachment of an internal staple of the peptide
DLXHLKXEKEKLLKEK

SEQ ID NO: 20 - protein, 16 aa, synthetic construct
  VARIANT 2: note = X is any unmodified amino acid and a site of attachment of an internal staple of the peptide
  VARIANT 6: note = X is any unmodified amino acid and a site of attachment of an internal staple of the peptide
DXDHLXDEKEKLLKEK

SEQ ID NO: 21 - protein, 16 aa, synthetic construct
  VARIANT 7: note = X is any unmodified amino acid and a site of attachment of an internal staple of the peptide
  VARIANT 11: note = X is any unmodified amino acid and a site of attachment of an internal staple of the peptide
DLDHLKXEKEXLLKEK

SEQ ID NO: 22 - protein, 16 aa, synthetic construct
  VARIANT 8: note = X is any unmodified amino acid and a site of attachment of an internal staple of the peptide
  VARIANT 12: note = X is any unmodified amino acid and a site of attachment of an internal staple of the peptide
DLDHLKDXKEKXLKEK

SEQ ID NO: 23 - protein, 16 aa, synthetic construct
  VARIANT 9: note = X is any unmodified amino acid and a site of attachment of an internal staple of the peptide
  VARIANT 13: note = X is any unmodified amino acid and a site of attachment of an internal staple of the peptide
DLDHLKDEXEKLXKEK

SEQ ID NO: 24 - protein, 16 aa, synthetic construct
  VARIANT 11: note = X is any unmodified amino acid and a site of attachment of an internal staple of the peptide
  VARIANT 15: note = X is any unmodified amino acid and a site of attachment of an internal staple of the peptide
DLDHLKDEKEXLLKXK

SEQ ID NO: 25 - skipped

SEQ ID NO: 26 - protein, 5 aa, synthetic construct
IVELE

SEQ ID NO: 27 - protein, 5 aa, synthetic construct
DLDHL

SEQ ID NO: 28 - protein, 6 aa, synthetic construct
DEKEKL

SEQ ID NO: 29 - protein, 6 aa, synthetic construct
LKDEKE

SEQ ID NO: 30 - protein, 6 aa, synthetic construct
KLLKEK

SEQ ID NO: 31 - protein, 5 aa, synthetic construct
ENDKS

(19) United States
(12) Patent Application Publication
(10) Pub. No.: US 2026/0365698 A1
(43) Pub. Date: Nov. 19, 2026

(54) STAPLED PEPTIDES FOR SENSITIZATION OF CANCER TO TREATMENT

(71) Applicant: Purdue Research Foundation, West Lafayette, IN (US)

(72) Inventors: Elizabeth Parkinson, West Lafayette, IN (US); Ramya Modi, Fremont, CA (US)

(73) Assignee: Purdue Research Foundation, West Lafayette, IN (US)

(21) Appl. No.: 19/137,098

(22) PCT Filed: Dec. 16, 2023
(86) PCT No.: PCT/US2023/084473

§ 371 Date: Jun. 9, 2025

Related U.S. Application Data
(60) Provisional application No. 63/433,206, filed on Dec. 16, 2022.

(51) Int. Cl. C07K 7/08; A61K 38/00

(52) U.S. Cl. CPC C07K 7/08; A61K 38/00; C07K 2319/60

(57) ABSTRACT

Stapled peptides and compositions thereof are provided that bind a musculoaponeurotic fibrosarcoma protein and thereby inhibit its heterodimerization with nuclear factor erythroid 2-related factor 2 (NRF2), a transcription factor that is upregulated in cancer cells. Methods of sensitizing a cancer to a cancer therapy are also provided, which use the stapled peptides and/or compositions hereof.

### PRIORITY

This application is related to and claims the priority benefit of U.S. Provisional Patent Application No. 63/433,206 filed Dec. 16, 2022. The content of the aforementioned application is hereby incorporated by reference in its entireties into this disclosure.

### TECHNICAL FIELD

The present disclosure relates to stapled peptides, pharmaceutical compositions, and a method of sensitizing cancer to therapy.

### STATEMENT OF SEQUENCE LISTING

A computer-readable form (CRF) of the Sequence Listing is submitted concurrently with this application, which is herein incorporated by reference in its entirety. The content of the computer-readable form is identical to the written sequence listing set forth herein.

SEQ ID NO: 1 is an amino acid sequence for Mafg: MGTSLTDEELVTMSVRELNQHLRGLSKEEIIQLKQRRRTLKNRGYAASCRVKRVTQKE ELEKQKAELQQEVEKLASENASMKLELDALRSKYE.

SEQ ID NO: 2 is an amino acid sequence for NRF2: HIPFPVEKIINLPVVDFNEMMSKEQFNEAQLALIRDIRRRGKNKVAAQNCRKRKLENIVE LEQDLDHLKDEKEKLLKEKGENDKSLHLLKKQL.

SEQ ID NO: 3 is an amino acid sequence for compound N1: IVELEQDLDHLKDEKE.

SEQ ID NO: 4 is an amino acid sequence for compound N2: DLDHLKDEKEKLLKEK.

SEQ ID NO: 5 is an amino acid sequence for compound N3: DEKEKLLKEKGENDKS.

SEQ ID NO: 6 is an amino acid sequence for compound NIS: IVELEXDLDXLKDEKE.

SEQ ID NO: 7 is an amino acid sequence for compound N2S: DLDHLXDEKXKLLKEK.

SEQ ID NO: 8 is an amino acid sequence for compound N3S: DEKEKLXKEKXENDKS.

SEQ ID NO: 9 is an amino acid sequence for compound N1SA: IVELXQDLXHLKDEKE.

SEQ ID NO: 10 is an amino acid sequence for compound N1SB: IVEXEQDXDHLKDEKE.

SEQ ID NO: 11 is an amino acid sequence for compound N1SC: IVXLEQXLDHLKDEKE.

SEQ ID NO: 12 is an amino acid sequence for compound N1SD: IVELEQXLDHXKDEKE.

SEQ ID NO: 13 is an amino acid sequence for compound N1SE: EVELEQDXDHLXDEKE.

SEQ ID NO: 14 is an amino acid sequence for compound N1SF: IVELEQDLXHLKXEKE.

SEQ ID NO: 15 is an amino acid sequence for compound N1SG: IVELEQDLDXLKDXKE.

SEQ ID NO: 16 is an amino acid sequence for compound NISH: IVELEXDLDHLKXEKE.

SEQ ID NO: 17 is an amino acid sequence for compound N2SA: DLDHXKDEXEKLLKEK.

SEQ ID NO: 18 is an amino acid sequence for compound N2SB: DLDXLKDXKEKLLKEK.

SEQ ID NO: 19 is an amino acid sequence for compound N2SC: DLXHLKXEKEKLLKEK.

SEQ ID NO: 20 is an amino acid sequence for compound N2SD: DXDHLXDEKEKLLKEK.

SEQ ID NO: 21 is an amino acid sequence for compound N2SE: DLDHLKXEKEXLLKEK.

SEQ ID NO: 22 is an amino acid sequence for compound N2SF: DLDHLKDXKEKXLKEK.

SEQ ID NO: 23 is an amino acid sequence for compound N2SG: DLDHLKDEXEKLXKEK.

SEQ ID NO: 24 is an amino acid sequence for compound N2SH: DLDHLKDEKEXLLKXK.

SEQ ID NO: 25 is an amino acid sequence of R₁X₂R₃X₄R₅, wherein R₁ is SEQ ID NO: 26, SEQ ID NO: 27, SEQ ID NO: 28, or has at least 80% sequence identity to SEQ ID NO: 26, SEQ ID NO: 27, or SEQ ID NO: 28; R₃ is DLD, DEK, KEK, or has at least 66% sequence identity to DLD, DEK, or KEK; R₅ is SEQ ID NO: 29. SEQ ID NO: 30. SEQ ID NO: 31, or is a functional variant of SEQ ID NO: 29. SEQ ID NO: 30, or SEQ ID NO: 31; and X₂ and X₄ are modified amino acids and are joined by an internal staple.

SEQ ID NO: 26 is the amino acid sequence IVELE.

SEQ ID NO: 27 is the amino acid sequence DLDHL.

SEQ ID NO: 28 is the amino acid sequence DEKEKL.

SEQ ID NO: 29 is the amino acid sequence LKDEKE.

SEQ ID NO: 30 is the amino acid sequence KLLKEK.

SEQ ID NO: 31 is the amino acid sequence ENDKS.

### BACKGROUND

Nuclear factor erythroid related 2-factor 2 (Nrf2) is a basic leucine zipper (bZip) transcription factor. It has a “cap ‘n’ collar” (CNC) structure and possesses seven highly conserved domains called NRF2-ECH homology (Neh) domains. The Neh1 domain allows Nrf2 to heterodimerize with small Maf proteins (e.g., MAFF, MAFG, and MAFK). The Neh2 domain allows for binding of NRF2 to the cytosolic repressor Keap1. The Neh3 domain may play a role in NRF2 protein stability and may act as a transactivation domain, interacting with a component of the transcriptional apparatus. The Neh4 and Neh5 domains act as transactivation domains and bind to the cAMP response element binding protein (CREB), which possesses intrinsic histone acetyltransferase activity. The Neh6 domain may contain a degron, which is involved in redox-insensitive degradation of NRF2. When the Neh7 domain associates with the retinoid X receptor a, it represses NRF2 transcriptional activity.

Under normal or unstressed conditions, NRF2 is kept in the cytoplasm by Kelch like-ECH-associated protein 1 (KEAP1) and Cullin 3 (Cul3), which degrade NRF2 by ubiquitination. Once ubiquitinated, NRF2 is transported to the proteasome, where it is degraded, and its components are recycled. Under normal conditions, NRF2 has a half-life of only 20 minutes.

Under oxidative or electrophilic stress, the Keap1-Cul3 ubiquitination system can be disrupted. NRF2 can build up in the cytoplasm and translocate to the nucleus, where it forms a heterodimer with a Maf protein and binds to the antioxidant response element (ARE) in the upstream promoter region of antioxidative genes, thereby initiating their transcription. NRF2 activation induces the transcription of genes encoding cytoprotective proteins, such as NAD(P)H quinone oxidoreductase (Nqo1), glutamate-cysteine ligase catalytic subunit (GCLC) and glutamate-cysteine ligase regulatory subunit (GCLM), sulfiredoxin 1 (SRXN1) and thioredoxin reductase 1 (TXNRD1), heme oxygenase-1 (HMOX1, HO-1), glutathione S-transferase (GST), UDP-glucuronosyltransferase (UGT), multidrug resistance-associated proteins (Mrps), and KEAP1.

NRF2 is ubiquitously expressed with the highest concentrations in the kidney, followed by muscle, lung, heart, liver, and brain. In normal cells, activation of NRF2 by a thiol-reactive stressor helps prevent (at least for a limited period of time) the inhibition of cancer by chemical carcinogens through induction of genes encoding drug-metabolizing enzymes. However, NRF2 is often overactivated. For example, in many tumor types. NRF2 is permanently upregulated. In such cases, its overexpressed target genes can support the promotion and progression of cancer by suppressing oxidative stress because they constitutively increase the capacity to scavenge reactive oxygen species (ROS) and support cell proliferation by increasing at least ribonucleotide synthesis, serine biosynthesis, and autophagy. Accordingly, the overactivation of NRF2 may promote the development of cancerous tumors de novo and lead to resistance to cancer treatment. By raising plasma cholesterol levels and cholesterol content in the liver, it may also promote the development of atherosclerosis.

Inhibitors of NRF2 have been developed. The best characterized examples are ML-385, AEM1, and Peptide 18. ML-385 binds to the Neh1 domain of NRF2 and interferes with the binding of the V-Maf avian musculoaponeurotic fibrosarcoma oncogene homologue G (MAFG)-NRF2 protein complex to regulatory DNA binding sequences, thereby inhibiting downstream target gene expression. Singh et al., Small molecule inhibitor of NRF2 selectively intervenes therapeutic resistance in KEAP1-deficient NSCLC tumors, ACS Chemical Biology 11(11): 3214-3225 (2016). When combined with platinum-based drugs, doxorubicin ortaxol, ML385 reportedly substantially enhances cytotoxicity in non-small cell lung cancer (NSCLC) cells. Id. AEM1 is a small molecule that decreases NRF2 transcriptional activity at antioxidant response element sites. Bollong et al., A small molecule inhibits deregulated NRF2 transcriptional activity in cancer, ACS Chemical Biology 10(10): 2193-2198 (2015). AEM1 reportedly sensitizes A549 cells (i.e., lung carcinoma epithelial cell line) to various chemotherapeutic agents and inhibits the growth of A549 cells: however, it appears that AEM1's activity is restricted to cell lines with mutations that render NRF2 constitutively active. Id.

Peptide 18 reportedly demonstrates high affinity, selective binding to the antioxidant response element (ARE) DNA. Simov et al., Discovery and characterization of novel peptide inhibitors of the NRF2/MAFG/DNA ternary complex for the treatment of cancer, European J Medicinal Chemistry 224: 113686 (2021). It displaces NRF2-MAFG from its promoter. Id.

The above NRF2 inhibitors are disadvantageous, however, whether due to lack of specificity, potency, or cell permeability issues. Thus, there is an unmet need for an NRF2 inhibitor that is specific and cell permeable. It is an object of the present disclosure to provide such an NRF2 inhibitor. This and other objects and advantages, as well as inventive features, will be apparent from the detailed description provided herein.

### SUMMARY

A stapled peptide is provided. The stapled peptide binds a musculoaponeurotic fibrosarcoma (sMAF) protein and thereby inhibits its heterodimerization with nuclear factor erythroid 2-related factor 2 (NRF2). The stapled peptide can be about 15 amino acids to about 20 amino acids in length, such as about 16 amino acids (e.g., 16 amino acids) in length. The stapled peptide can bind MAFG, such as with a kD of about 335 to about 340 nM. The stapled peptide can sensitize Nrf2-addicted cancer to cisplatin. The stapled peptide can be NIS (Ac-IVELEXDLDXLKDEKE-NH₂ (SEQ ID NO: 6), in which the ends of the staple are positioned at the Xs).

In certain embodiments, a stapled peptide, which binds sMAF protein and thereby inhibits its heterodimerization with NRF2 is provided. The stapled peptide can comprise a peptide chain comprising at least two modified amino acids joined by at least one internal staple. In certain embodiments, the stapled peptide comprises a single staple.

The peptide chain can be about 15 amino acids to about 20 amino acids (e.g., 15-20 amino acids). The peptide chain can be 16 amino acids in length. The peptide chain can comprise a cell penetrating peptide.

In certain embodiments, the stapled peptide can bind MafG with a kD of about 335 to about 340 nM (e.g., 335-340 nM). In certain embodiments, the stapled peptide can sensitize Nrf2-addicted cancer to cisplatin.

The stapled peptide can comprise SEQ ID NO: 6 or a functional variant thereof, wherein each X of SEQ ID NO: 6 is a modified amino acid. The stapled peptide can comprise SEQ ID NO: 7 or a functional variant thereof, wherein each X of SEQ ID NO: 7 is a modified amino acid. The stapled peptide can comprise SEQ ID NO: 8 or a functional variant thereof, wherein each X is a modified amino acid. In certain embodiments, each X of a sequence hereof is or comprises (S)-2-amino-2-methyl-hept-6-enoic acid. The ends of the internal staple of each peptide can connect at each X.

In certain embodiments, the stapled peptide comprises SEQ ID NO: 6, SEQ ID NO: 7, SEQ ID NO: 8, or a functional variant of SEQ ID NO: 6, SEQ ID NO: 7, or SEQ ID NO: 8, wherein each X of the respective sequence is or comprises (S)-2-amino-2-methyl-hept-6-enoic acid. The ends of the at least one internal staple of each peptide can connect at each X. The at least one internal staple can be at positions [i, i+4] of the peptide chain, wherein “i” represents the first staple position in the peptide chain.

The stapled peptide can be cell-permeable.

The at least one internal staple can be a hydrocarbon tether. The at least one internal staple can be a hydrocarbon tether and comprise about 8 to about 10 carbon atoms (e.g., 8-10 carbon atoms). In certain embodiments, the hydrocarbon staple is pegylated. In certain embodiments, the at least one internal staple comprises an olefin tether. In certain embodiments, the at least one internal staple comprises a macrocyclic tether. In certain embodiments, the at least one internal staple comprises about 6 to about 16 carbon atoms (e.g., 6-16 carbon atoms). The at least one internal staple can comprise 8 carbon atoms. The at least one internal staple can be substituted with at least one alkyl. The at least one alkyl can comprise a methyl.

The stapled peptide can comprise a helical conformation and, optionally, an α-helical conformation. In certain embodiments, the stapled peptide can further comprise a tracer (e.g., is linked to a tracer). The tracer can comprise a fluorescent molecule.

In certain embodiments, the stapled peptide is modified by acetylation at a 5′-terminus thereof.

The stapled peptide can comprise the amino acid sequence of R₁X₂R₃X₄R₅ (SEQ ID NO: 25), wherein:
R₁ is SEQ ID NO: 26, SEQ ID NO: 27, SEQ ID NO: 28, or has at least 80% sequence identity to SEQ ID NO: 26, SEQ ID NO: 27, or SEQ ID NO: 28;
R₃ is DLD, DEK, KEK, or has at least 66% sequence identity to DLD, DEK, or KEK;
R₅ is SEQ ID NO: 29, SEQ ID NO: 30, SEQ ID NO: 31, or is a functional variant of SEQ ID NO: 29, SEQ ID NO: 30, or SEQ ID NO: 31; and
X₂ and X₄ are modified amino acids joined by a first internal staple.

The amino acid sequence can be selected from the group consisting of SEQ ID NO: 6, SEQ ID NO: 7, and SEQ ID NO: 8. The first internal staple can comprise the only staple of the stapled peptide. The first internal staple can comprise a hydrocarbon tether. The first internal staple can comprise an olefin tether. The first internal staple can comprise a macrocyclic tether.

In certain embodiments, each X₂ and X₄ is or comprises (S)-2-amino-2-methyl-hept-6-enoic acid.

In certain embodiments, the stapled peptide (e.g., comprising SEQ ID NO: 25) comprises a helical conformation and, optionally, an α-helical conformation.

One or more of the amino acids of R₁, R₃, and/or R₅ of SEQ ID NO: 25 can have a D-configuration. One or more of the amino acids of R₁, R₃, and/or R₅ of SEQ ID NO: 25 can have an L-configuration. The at least one internal staple can be at positions [i, i+4] of the peptide chain, wherein “i” represents the first staple position in the peptide chain.

In certain embodiments, SEQ ID NO: 25 is linked to or further comprises a cell penetrating peptide.

A pharmaceutical composition is further provided. The composition can comprise any of the stapled peptides hereof and a pharmaceutically acceptable carrier and/or diluent.

A method of sensitizing a cancer to a cancer therapy is still further provided. The method can comprise contacting a cancer cell with a cancer therapy-sensitizing amount of a stapled peptide hereof or a pharmaceutical composition hereof comprising any of the stapled peptides hereof and a pharmaceutically acceptable carrier and/or diluent, whereupon the cancer cell is sensitized to the cancer therapy. The cancer can be Nrf2-addicted. The cancer therapy can comprise the administration of a chemotherapy agent. The cancer therapy can comprise the administration of cisplatin. The cancer can be cancer of the lung, breast or pancreas. The lung cancer can be non-small cell lung cancer. The cancer therapy can comprise an immune checkpoint inhibitor, an immune modulator, or a combination of both of the foregoing.

In certain embodiments, the method further comprises subjecting the cancer cell to radiation therapy, surgery, thermos-ablation, focused ultrasound therapy, cryotherapy, or any combination of the foregoing.

Uses of any of the stapled peptides hereof are also provided. In certain embodiments, the use comprises a use of any of the stapled peptides hereof or the pharmaceutical composition hereof in the preparation of a medicament for the sensitization of a cancer to a cancer therapy. The cancer can be lung cancer, breast cancer, or pancreatic cancer. The cancer can be non-small cell lung cancer. The cancer therapy can comprise administration of a chemotherapeutic agent to a subject.

The chemotherapeutic agent can be cisplatin.

### BRIEF DESCRIPTION OF THE FIGURES

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1A shows ZIP domains of nuclear factor erythroid related 2-factor 2 (Nrf2) and V-Maf avian musculoaponeurotic fibrosarcoma oncogene homologue G (MafG) with key interacting residues indicated in bold.

FIG. 1B shows an AlphaFold model of Nrf2-MafG heterodimer, with the top left image showing the AlphaFold Model, the top right image showing a close up of the portion of the top left image within the rectangle, and the bottom image showing a spacefilling close up of the portion of the top left image within the rectangle. Peptides covered by stapled peptides are indicated in the darkest shading (see, e.g., darkest shading in the top left image within the rectangle). Staple locations are circled (top right and lower images).

FIG. 1C shows unstapled (compounds N1, N2, and N3) and stapled peptides (compounds NIS, N2S, and N3S).

FIG. 2 shows the AlphaFold model of Nrf2 and MafG with target Nrf2 peptides indicated, with the top row showing them at the same angle, the middle row showing them rotated to the best angle, and the bottom row showing spacefilling at the best angle. M=Mafg; A=Nrf2; B and C=key interacting residues, respectively; D=the peptide of interest (compound N1. N2, or N3); and the location of the staple is circled.

FIG. 3A shows circular dichroism spectra, which demonstrate that the stapled peptides are more α-helical compared to their non-stapled controls.

FIG. 3B shows the results of protease assays in which 55 M peptide was incubated with 10 μg/mL proteinase K for the indicated timepoints. N3 unstapled peptide was completely degraded at the shortest timepoint (30 minutes) and not included. Data are from three independent replicates with standard error indicated. Scale bar indicates 50 μm.

FIG. 4A shows the results of treatment of HEK-293 cells with NQO1-ARE firefly luciferase and TK-Renilla luciferase reporters treated with compound (50 μM) or vehicle control. The results are averages from three replicates.

FIG. 4B shows the dose response for N1S in the luciferase assay. The results are averages from two replicates.

FIG. 5A shows luciferase and Renilla data from three replicates. *p<0.1, **p<0.01, ***p<0.001, student's T-test.

FIG. 5B shows N1S and N2S derivatives.

FIG. 5C shows screening data for N1S and N2S derivatives.

FIG. 6A shows the results of a split luciferase assay with purified MafG and Nrf2. Data shown are from three technical replicates with standard error. The data are representative of three independent experiments. *p<0.05, **p<0.01, ***p<0.001, student's T-test compared to dimethyl sulfoxide (DMSO).

FIG. 6B shows an equilibrium binding response curve using the one-site binding model generated from the SPr data. Data are from three independent experiments with standard error.

FIG. 6C is a Western blot for expression levels of TrxR1 by A549 cells treated with various concentrations of N1S or a DMSO control. Data are from three independent experiments.

FIG. 6D shows quantification of Trx1 expression levels relative to actin. Data are from three independent experiments with standard deviation. Quantification was performed using ImageJ.

FIG. 6E shows GSH quantification from one-hour treatment of A549 cells with the indicated concentrations of N1 S or a DMSO control. Data are from three independent experiments with standard deviation. ****p<0.0001, one-way ANOVA.

FIG. 7A shows the results of a MTT assay after treatment with NTS, cisplatin, or both after 72 hours. Percent dead in red color indicates higher percentage of death. Data are representative of three independent replicates.

FIG. 7B shows the combination index (CI) as calculated with CompuSyn for the data shown in FIG. 7A. Cl values<1 were synergistic (red), CI values=1 were additive (green), and CI values>1 were antagonistic (not observed).

FIG. 7C shows the data from Incucyte Live-Cell Analysis of treatment of cells with vehicle (DMSO). NTS, cisplatin, or a combination thereof. Error bars represent the standard deviation between three technical replicates. The data are representative of three independent experiments.

While the present disclosure is susceptible to various modifications and alternative forms, exemplary embodiments thereof are shown by way of example in the drawings and are herein described in detail.

### DETAILED DESCRIPTION

While the concepts of the present disclosure are illustrated and described in detail in the description herein, results in the description are to be considered as exemplary and not restrictive in character; it being understood that only the illustrative embodiments are shown and described and that all changes and modifications that come within the spirit of the disclosure are desired to be protected.

The present disclosure is predicated, at least in part, on the discovery that a stapled peptide (i.e., a short α-helical peptide stabilized by a hydrocarbon staple) can inhibit nuclear factor erythroid 2-related factor 2 (NRF2) heterodimerization with musculoaponeurotic fibrosarcoma (sMAF) proteins in the nucleus, thereby inhibiting binding of the heterodimer to DNA and induction of transcription of NRF2-regulated genes. Stapled peptides can target large surface area interactions, such as that presented by NRF2-MAFG, and can specifically inhibit protein-protein interactions. These cell-permeable peptides are structurally stabilized, orally bioavailable and have increased resistance to proteolysis and enhanced alpha helicity.

Approximately 90% of failures in chemotherapy treatments are due to drug resistance. While many people are exploring the development of novel chemotherapeutics to overcome this resistance, another more direct method is to inhibit the mechanisms of resistance. Nrf2, for example, is often constitutively activated in solid tumors, and many of its downstream genes are responsible for chemotherapy resistance.

The present disclosure provides direct inhibitors of heterodimerization of Nrf2 with its partner MafG. Specifically, AlphaFold models were used to design stapled peptides to inhibit heterodimerization, and a promising lead inhibitor (N1S), which can enter cells and inhibit Nrf2 in luciferase reporter assays, was identified. In vitro analysis, including fluorescence polarization, split-luciferase, immunoprecipitation, and SPR, demonstrates that NIS directly binds MafG and inhibits its dimerization with Nrf2. Finally, studies of N1S in combination with cisplatin demonstrated strong synergy against A549 non-small cell lung cancer cells. Overall, the potential of utilizing NIS as an inhibitor of Nrf2/MafG signaling to sensitize cancers to traditional chemotherapeutics has been demonstrated.

In view of the above, a stapled peptide is provided. When administered, the stapled peptide can bind to a sMAF protein and thereby inhibit its heterodimerization with a transcription factor that is upregulated in cancer cells. As used herein, “sMAF” includes all family members, mutants, alleles, fragments, species, coding and noncoding sequences, sense and antisense polynucleotide strands, etc. Aliases of sMAF include MAF BZIP transcription factor, C-MAF, V-Maf avian musculoaponeurotic fibrosarcoma oncogene homolog, transcription factor Maf, proto-oncogene C-Maf, avian musculoaponeurotic fibrosarcoma protooncogene, V-Maf musculoaponeurotic fibrosarcoma oncogene homolog, T lymphocyte C-Maf long form. C-Maf proto-oncogene, CTRCT21, AYGRP, and CCA4.

The stapled peptide can bind MafG. In certain embodiments, the stapled peptide can bind MafG with a kD of about 335 to about 340 nM (such as about 335 nm to 340 nm, 335 nm to 340 nm, or 335 nm to about 340 nm).

The stapled protein can comprise a peptide chain comprising an external brace that forces the peptide structure into an α-helical structure. In certain embodiments, the stapled peptide comprises at least two modified amino acids joined by at least one internal (intramolecular) cross-link (or staple).

The stapled peptides described herein include stapled peptides, as well as peptides structurally reinforced by other chemical strategies known in the art. As used herein, “peptide stapling” is a term coined from a synthetic methodology wherein two side-chains (e.g., cross-linkable side chains) present in a polypeptide chain are covalently joined (e.g., “stapled together”) using a ring-closing metathesis (RCM) reaction to form a cross-linked ring. The term “peptide stapling” includes, for example, the joining of two (e.g., at least one pair of) double bond-containing side-chains, triple bond-containing side-chains, or double bond-containing and triple bond-containing side chains, which can be present in a polypeptide chain, using any number of reaction conditions and/or catalysts to facilitate such a reaction to provide a singly “stapled” polypeptide. In some instances, staples can retain the unsaturated bond or can be reduced.

Stapling allows a peptide to maintain a constrained or discrete three-dimensional structure or ensemble of structures. The stapled peptide can increase hydrophobicity, cell permeability, and protease resistance. In some embodiments, the stapled peptide has a helical conformation (e.g., an alpha helix).

In certain embodiments, the stapled peptides hereof can be any stabilized peptides that are permeable to cell membranes (e.g., enter the cell). In certain embodiments, the stapled peptides comprise at least one internal staple and a peptide chain comprising at least four amino acid groups.

In certain embodiments, the peptide chain can be about 15 amino acids to about 20 amino acids in length, such as about 15 amino acids (e.g., 15 amino acids) in length, such as about 16 amino acids (e.g., 16 amino acids) in length, such as about 17 amino acids (e.g., 17 amino acids) in length, such as about 18 amino acids (e.g., 18 amino acids) in length, such as about 19 amino acids (e.g., 19 amino acids) in length, or such as about 20 amino acids (e.g., 20 amino acids) in length.

The peptide chain can comprise a cell penetrating peptide. Cell penetrating peptides can be, for example, a short peptide of less than about 30 (such as less than 30) amino acids either derived from proteins or from chimeric sequences. They can be amphipathic and possess a net positive charge.

Cell penetrating peptides are able to penetrate biological membranes, to trigger the movement of various biomolecules across cell membranes into the cytoplasm and to improve their intracellular routing. As such, inclusion of a cell penetrating peptide within the peptide chain of the stapled peptide can facilitate or otherwise improve interactions of with a targeted cell.

The cell penetrating peptide can be any cell penetrating peptide known in the art or hereinafter developed. In certain embodiments, the cell penetrating peptide is or comprises a Tat peptide, a penetratin, an arginine-rich peptide, a transportan, a polyarginine peptide (e.g., R8 or R9), or a peptide-derived from a VP22 protein.

The at least one internal staple of the stapled peptide can be a hydrocarbon tether. The hydrocarbon tether can comprise about 8 to about 20 carbons. The hydrocarbon tether can comprise about 10 to about 18 carbons. The hydrocarbon tether can comprise about 12 to about 16 carbons. The hydrocarbon tether can comprise about 14 carbons. The hydrocarbon tether can comprise 8 carbons. The ranges set forth in this paragraph are inclusive of the stated end points and include all 1 carbon increments within each stated range.

The hydrocarbon tether can be methylated. The hydrocarbon tether can be demethylated. In certain embodiments, the at least one internal staple is pegylated.

In certain embodiments, the at least one internal staple comprises an olefin tether. The olefin tether can comprise 8 carbons. The olefin tether can comprise 10 carbons. The olefin tether can comprise more than 8 carbons.

Each internal staple can comprise about 6 to about 16 carbon atoms. The internal staple can comprise about 8 to about 14 carbons. The internal staple can comprise about 10 to about 12 carbons. The internal staple can comprise 8 carbons. The ranges set forth in this paragraph are inclusive of the stated end points and include all 1 carbon increments within each stated range.

In certain embodiments, the internal staple is at (or spans) positions [i, i+4] of the stapled peptide. In certain embodiments, the internal staple is at (or spans) positions [i, i+7] of the stapled peptide. As used herein “i” represents the first staple position in the peptide. In certain embodiments, the internal staple is at position R5, R8, and/or S8 of the peptide pursuant to concepts described in Walensky & Bird, Hydrocarbon-stapled peptides: principles, practice, and progress, J Medical Chemistry 57(15): 6275-6288 (2014). In certain embodiments, S8-R5 are substituted at positions [i, i+7] or positions [i, i+4] to generate a single-stapled peptide. In certain embodiments, R8 is substituted at positions [i, i+7] or positions [i, i+4] to generate a single-stapled peptide.

The internal staple can be optionally substituted. In certain embodiments, the internal staple is substituted with at least one alkyl. The at least one alkyl can comprise a methyl group. In certain embodiments, the internal staple comprises at least two methyl substituents.

In certain embodiments, the internal staple comprises a macrocyclic tether. As used herein, “macrocyclic” means more than about 12 atoms in the ring.

The stapled peptide can comprise or be SEQ ID NO: 6 or a functional variant thereof, wherein each X of SEQ ID NO: 6 is a modified amino acid and represents an attachment site of an end of the internal staple of the stapled peptide. The term “functional variant” refers to a peptide, a polypeptide, or a protein having substantial or significant sequence identity or similarity to the reference peptide or polypeptide, which functional variant retains the biological activity of the reference peptide or polypeptide of which it is a variant. Functional variants encompass, for example, those variants of the peptide chain (the parent peptide) that retain the ability to exhibit the properties (such as, for example, helical structure, cell permeability, oral bioavailability, and/or cancer sensitizing ability) and to a similar extent, the same extent, or to a higher extent, as the parent peptide. In reference to a nucleic acid sequence encoding the peptide, in some embodiments a nucleic acid sequence encoding a functional variant of the peptide or is about 10% identical, about 25% identical, about 30% identical, about 50% identical, about 65% identical, about 75% identical, about 80% identical, about 90% identical, about 95% identical, or about 99% identical to the nucleic acid sequence encoding the parent peptide.

The term “modified amino acid” as used herein means an amino acid that is not a protein constituent amino acid or a post-translationally modified variant thereof. In particular, modified amino acids can be derived forms of proteinogenic amino acids.

In certain embodiments, the stapled peptide comprises SEQ ID NO: 7 or a functional variant thereof, wherein each X of SEQ ID NO: 7 is a modified amino acid and represents an attachment site of an end of the internal staple of the stapled peptide. In certain embodiments, the stapled peptide comprises SEQ ID NO: 8 or a functional variant thereof, wherein each X is a modified amino acid and represents an attachment site of an end of the internal staple of the stapled peptide.

In certain embodiments, the stapled peptide comprises SEQ ID NO: 9 or a functional variant thereof, wherein each X is a modified amino acid and represents an attachment site of an end of the internal staple of the stapled peptide. In certain embodiments, the stapled peptide comprises SEQ ID NO: 10 or a functional variant thereof, wherein each X is a modified amino acid and represents an attachment site of an end of the internal staple of the stapled peptide. In certain embodiments, the stapled peptide comprises SEQ ID NO: 11 or a functional variant thereof, wherein each X is a modified amino acid and represents an attachment site of an end of the internal staple of the stapled peptide. In certain embodiments, the stapled peptide comprises SEQ ID NO: 12 or a functional variant thereof, wherein each X is a modified amino acid and represents an attachment site of an end of the internal staple of the stapled peptide. In certain embodiments, the stapled peptide comprises SEQ ID NO: 13 or a functional variant thereof, wherein each X is a modified amino acid and represents an attachment site of an end of the internal staple of the stapled peptide. In certain embodiments, the stapled peptide comprises SEQ ID NO: 14 or a functional variant thereof, wherein each X is a modified amino acid and represents an attachment site of an end of the internal staple of the stapled peptide. In certain embodiments, the stapled peptide comprises SEQ ID NO: 15 or a functional variant thereof, wherein each X is a modified amino acid and represents an attachment site of an end of the internal staple of the stapled peptide. In certain embodiments, the stapled peptide comprises SEQ ID NO: 16 or a functional variant thereof wherein each X is a modified amino acid and represents an attachment site of an end of the internal staple of the stapled peptide. In certain embodiments, the stapled peptide comprises SEQ ID NO: 17 or a functional variant thereof, wherein each X is a modified amino acid and represents an attachment site of an end of the internal staple of the stapled peptide. In certain embodiments, the stapled peptide comprises SEQ ID NO: 18 or a functional variant thereof, wherein each X is a modified amino acid and represents an attachment site of an end of the internal staple of the stapled peptide. In certain embodiments, the stapled peptide comprises SEQ ID NO: 19 or a functional variant thereof, wherein each X is a modified amino acid and represents an attachment site of an end of the internal staple of the stapled peptide. In certain embodiments, the stapled peptide comprises SEQ ID NO: 20 or a functional variant thereof, wherein each X is a modified amino acid and represents an attachment site of an end of the internal staple of the stapled peptide. In certain embodiments, the stapled peptide comprises SEQ ID NO: 21 or a functional variant thereof, wherein each X is a modified amino acid and represents an attachment site of an end of the internal staple of the stapled peptide. In certain embodiments, the stapled peptide comprises SEQ ID NO: 22 or a functional variant thereof, wherein each X is a modified amino acid and represents an attachment site of an end of the internal staple of the stapled peptide. In certain embodiments, the stapled peptide comprises SEQ ID NO: 23 or a functional variant thereof, wherein each X is a modified amino acid and represents an attachment site of an end of the internal staple of the stapled peptide. In certain embodiments, the stapled peptide comprises SEQ ID NO: 24 or a functional variant thereof, wherein each X is a modified amino acid and represents an attachment site of an end of the internal staple of the stapled peptide.

The modified amino acid of any of the aforementioned sequences can comprise any modified or otherwise unnatural amino acid known in the art. Each X of a sequence set forth herein can be the same modified amino acid or a different modified amino acid. In certain embodiments, each X (e.g., the modified amino acid) of a sequence hereof can be independently selected from the group consisting of 2-naphthyl-alanine, statine, t-leucine, a peptoid derivative, a homo-amino acid or derivative thereof, a γ-amino acid, and an N-alkylated derivative. In certain embodiments, each X of a sequence hereof can be independently selected from the group consisting of hydroxyproline, lanthionine, 2-aminoisobutyric acid, deydroalanine, gamma-aminobutyric acid, ortnithine, citrulline, beta alanine (3-aminopropanoic acid), gamma-carboxyglutamate, selenocysteine, pyrrolysine. N-formylmethionine, 5-hydroxytryptophan, L-dihydroxyphenylalanine, triiodothyronine, L-3,4-dihydroxyphenylalainen (DOPA), and O-phosphoserine.

In certain embodiments, each X is or comprises 2-amino-2-methyl-hept-6-enoic acid. In certain embodiments, each X is or comprises (S)-2-amino-2-methyl-hept-6-enoic acid.

In certain embodiments, the stapled peptide comprises an amino acid sequence of R₁X₂R₃X₄R₅ (SEQ ID NO: 25), wherein:
R₁ is SEQ ID NO: 26. SEQ ID NO: 27, SEQ ID NO: 28, or has at least about 80% sequence identity (such as 75%-99% sequence identity) to SEQ ID NO: 26, SEQ ID NO: 27, or SEQ ID NO: 28;
R₃ is DLD, DEK, KEK, or has at least about 66% sequence identity (such as 60%-99% sequence identity) to DLD, DEK, or KEK;
R₅ is SEQ ID NO: 29, SEQ ID NO: 30, SEQ ID NO: 31, or is a functional variant of SEQ ID NO: 29, SEQ ID NO: 30, or SEQ ID NO: 31; and
X₂ and X₄ are each independently a modified amino acid (e.g., an unnatural amino acid) and are joined by an internal staple.

In certain embodiments, the stapled peptide comprises SEQ ID NO: 6, SEQ ID NO: 7, SEQ ID NO: 8, or a functional variant of SEQ ID NO: 6, SEQ ID NO: 7, or SEQ ID NO: 8, wherein each X of the respective sequence is a modified amino acid and represents an attachment site of an end of the internal staple of the stapled peptide. In certain embodiments, each X (i.e., modified amino acid) is or comprises 2-amino-2-methyl-hept-6-enoic acid and the staple is at (or spans) positions [i, i+4], wherein “i” represents the first staple position in the peptide.

Each of SEQ ID NOS: 6-8 can comprise the same modified amino acids therein, or the modified amino acids thereof can be different. In certain embodiments, each modified amino acid of SEQ ID NOS: 6-8 is or comprises (S)-2-amino-2-methyl-hept-6-enoic acid. In certain embodiments, each modified amino acid of SEQ ID NOS: 6-8 is or comprises (S)-2-amino-2-methyl-hept-6-enoic acid and the staple is at (or spans) positions [i, i+4], “i” represents the first staple position in the peptide.

In certain embodiments, the stapled peptides comprise a tracer (e.g., a fluorescent molecule such as TAMRA, FITC, etc.). Such molecules can be used to assess cellular uptake of the stapled peptide.

The stapled peptide can be further modified by acetylation. In certain embodiments, a 5′-terminus of the stapled peptide is acetylated.

In certain embodiments, the peptide chain of the stapled peptide comprises a combination of modified amino acids and naturally occurring amino acids. The peptide chain can, for example, comprise four to 16 natural amino acids selected from isoleucine, valine, glutamic acid, leucine, aspartic acid, and lysine. In certain embodiments, the peptide chain can comprise four to 16 natural amino acids selected from isoleucine, valine, glutamic acid, leucine, aspartic acid, lysine, and asparagine. One of more of the amino acids of the peptide chain can have a D-configuration. One or more of the amino acids of the peptide chain can have an L-configuration.

The stapled peptide can be compound NIS (Ac-IVELEXDLDXLKDEKE-NH₂, in which the ends of the staple are positioned at the Xs).

The stapled peptide can bind a sMAF protein and thereby inhibit its heterodimerization with NRF2. As used herein. “NRF2” includes all family members, mutants, alleles, fragments, species, coding and noncoding sequences, sense and antisense polynucleotide strands, etc. Aliases of NRF2 include NFE2L2, NFE2 like bZIP transcription factor 2, nuclear factor erythroid 2 like 2, NF-E2-related factor 2, NRF-2, HEBP1, Nrf-2, nuclear factor (erythroid-derived 2)-like 2, nuclear factor erythroid derived 2 like 2, nuclear factor erythroid-derived 2-like 2, nuclear factor erythroid 2-like 2, NFE2-related factor 2, and IMDDHH.

The stapled peptide can sensitize NRF2-addicted cancer to a chemotherapeutic agent. The chemotherapeutic agent can be or comprise cisplatin. The chemotherapeutic agent can be or comprise an alkylating agent, an antimetabolite, an anthracycline, an antitumor antibiotic, a platinum-based chemotherapeutic agent, a chemotherapeutic agent, or a plant alkaloid. The chemotherapeutic agent can be oxaliplatin, cyclophosphamide, ifosfamide, thiotepa, melphalan, busulfun, nimustine, ranimustine, dacarbazine, procarbazine, temozolomide, cisplatin, carboplatin, nedaplatin, methotrexate, pemetrexed, fluorouracil, tegaful/uracil, doxifluridine, tegaful/gimeracil/oteracil, capecitabine, cytarabine, enocitabine, gemcitabine, 6-mercaptopurine, fuludarabin, pentostatin, cladribine, hydroxyurea, doxorubicin, epirubicin, daunorubicin, idarubicine, pirarubicin, mitoxantrone, amurubicin, actinomycin D, bleomycin, pepleomycin, mitomycin C, aclarubicin, zinostatin, vincristine, vindesine, vinblastine, vinorelbine, paclitaxel, docetaxel, irinotecan, irinotecan active metabolite (SN-38), nogitecan (topotecan), etoposide, prednisolone, dexamethasone, tamoxifen, teremifene, medroxyprogesterone, anastrozole, exemestane, letrozole, rituximab, imatinib, gefitinib, gemtuzumab ozogamicin, bortezomib, erlotinib, cetuximab, bevacizumab, sunitinib, sorafenib, dasatinib, panitumumab, asparaginase, tretinoin, arsenic trioxide, or a salt, a hydrate, or a solvate of any of the foregoing, as well as any combination thereof.

Methods of synthesizing the peptides described herein are known in the art. Nevertheless, some methods of synthesis are exemplified herein. It will be appreciated that the various steps may be performed in an alternate sequence or order to give the desired compounds. Synthetic chemistry transformations and protecting group methodologies (protection and deprotection) useful in synthesizing the compounds described herein are known in the art and include, for example, those such as described in Larock, Comprehensive Organic Transformations, VCH Publishers (1989); Greene & Wuts, Protective Groups in Organic Synthesis, John Wiley & Sons (1995), and subsequent editions thereof. Again, methods suitable for obtaining (e.g., synthesizing), stapling, and purifying the peptides hereof are known in the art.

In certain embodiments, the peptides are substantially free of non-stapled peptide contaminants or are isolated. Methods for purifying peptides include, for example, synthesizing the peptide on a solid-phase support.

A pharmaceutical composition is further provided. In certain embodiments, the composition comprises any of the stapled peptides described herein and one or more pharmaceutically acceptable carriers, diluents, and/or other pharmaceutically acceptable components. The carriers, diluents, and/or other components can be determined in part by the particular route of administration (see, e.g., Remington's Pharmaceutical Sciences, 17^{th} ed. (1985)). The ingredients of the composition can be of sufficiently high purity and sufficiently low toxicity such that the composition is suitable for administration to a human. The composition desirably is stable. “Pharmaceutically acceptable carrier” is used herein as an umbrella term to cover the inclusion of carriers, diluents and/or other pharmaceutically acceptable components.

The choice of carrier will be determined in part by the particular stapled peptides, as well as by the particular method used to administer the peptides. Accordingly, there are a variety of suitable formulations of the pharmaceutical composition. For example, the pharmaceutical composition can contain preservatives. Suitable preservatives can include, for example, methylparaben, propylparaben, sodium benzoate, and benzalkonium chloride. A mixture of two or more preservatives optionally can be used. The preservative or mixtures thereof are typically present in an amount of about 0.0001% to about 2% by weight of the total composition.

The particular formulation employed can, as noted above, also depend on the particular route of administration desired. For example, a formulation suitable for systemic, e.g., intravenous, administration, may differ from a formulation suitable for intracranial administration. Such modifications are within the ordinary skill in the art.

A method of sensitizing a cancer to a cancer therapy is still further provided. “Cancer” includes any neoplastic condition, whether malignant, pre-malignant or non-malignant. Generally, however, the neoplastic condition is malignant. Both solid and non-solid tumors are encompassed, and “cancer(ous) cell” may be used interchangeably with “tumor(ous) cell.”

When a cancer involves excess NRF2, whether due to lack of degradation, reduced degradation, constitutive activation, or other over-expression, intrinsic resistance to anti-cancer therapies (such as chemotherapy and radiotherapy) and aggressive proliferation result, and overall survival is poor. Examples of such cancers include, but are not limited to, lung cancer, such as non-small cell lung cancer (NSCLC: e.g., squamous NSCLC), breast cancer, and pancreatic cancer. Indeed, 25% of lung cancers have been reported to have mutations in Kelch like-ECH-associated protein 1 (KEAP1), which along with Cullin 3 (Cul3), degrade NRF2 in the cytoplasm by ubiquitination (Giordano et al., Trends Molec Med 22: 578-593 (2016); and Motohashi et al., Cancer Cell 22: 66-79 (2012)). KEAP1 mutations are also reported for 37% of ovarian carcinomas and 31% of gallbladder carcinomas; many others have mutations in KEAP1 or NRF2.

In certain embodiments, the method comprises contacting the cancer (e.g., cancer cells) with a cancer therapy-sensitizing amount of a stapled peptide hereof or a pharmaceutical composition comprising the same and a pharmaceutically acceptable carrier. “Cancer therapy-sensitizing amount,” as used herein, means an amount of a stapled peptide (or a pharmaceutical composition comprising the stapled peptide) that sensitizes the cancer to the cancer therapy. In other words, the administration of the stapled peptide helps the cancer therapy at least additively, if not synergistically, inhibit the progression of the cancer (e.g., proliferation of cancerous cells and/or the metastasis thereof). Desirably, the stapled peptide helps the cancer therapy at least additively, if not synergistically, to kill cancerous cells such that the number of cancerous cells decreases, desirably to the point of eradication. The cancer can be Nrf2-addicted.

The stapled peptide can be administered with a chemotherapeutic agent, such as an alkylating agent, an antimetabolite, an anthracycline, an antitumor antibiotic, a platinum-based chemotherapeutic agent, a chemotherapeutic agent, or a plant alkaloid. The chemotherapeutic agent can be oxaliplatin, cyclophosphamide, ifosfamide, thiotepa, melphalan, busulfun, nimustine, ranimustine, dacarbazine, procarbazine, temozolomide, cisplatin, carboplatin, nedaplatin, methotrexate, pemetrexed, fluorouracil, tegaful/uracil, doxifluridine, tegaful/gimeracil/oteracil, capecitabine, cytarabine, enocitabine, gemcitabine, 6-mercaptopurine, fuludarabin, pentostatin, cladribine, hvdroxvurea, doxorubicin, epirubicin, daunorubicin, idarubicine, pirarubicin, mitoxantrone, amurubicin, actinomycin D, bleomycin, pepleomycin, mitomycin C, aclarubicin, zinostatin, vincristine, vindesine, vinblastine, vinorelbine, paclitaxel, docetaxel, irinotecan, irinotecan active metabolite (SN-38), nogitecan (topotecan), etoposide, prednisolone, dexamethasone, tamoxifen, teremifene, medroxyprogesterone, anastrozole, exemestane, letrozole, rituximab, imatinib, gefitinib, gemtuzumab ozogamicin, bortezomib, erlotinib, cetuximab, bevacizumab, sunitinib, sorafenib, dasatinib, panitumumab, asparaginase, tretinoin, arsenic trioxide, or a salt, a hydrate, or a solvate of any of the foregoing, as well as any combination thereof.

The stapled peptide, alone or in further combination with a chemotherapeutic agent, can be further administered with an immune checkpoint inhibitor, an immune modulator, or a combination of both. The immune checkpoint inhibitor can inhibit or block CTLA-4, PD-1, PD-L1. LAG-3, GITR, and/or TIM3, such as an antibody. The immune modulator can be an interleukin, interferon, tumor necrosis factor (TNF) or other growth factor, colony stimulating factor, T cell modulator, cytokine, or hormone, which stimulates the immune response, or a salt, a hydrate, or a solvate of any of the foregoing, as well as any combination thereof.

The stapled peptide and the chemotherapeutic agent, alone or in further combination with an immune checkpoint inhibitor, an immune modulator, or both, can be administered simultaneously or sequentially, in any order, by the same or different routes. Such routes include, but are not limited to, oral, intravenous, and intratumoral. The formulation of compositions suitable for administration by such routes is within the ordinary skill in the art (see, e.g., Remington's Pharmaceutical Sciences, 17^{th} ed. (1985)).

The cancer therapy can further comprise radiation therapy, surgery, thermos-ablation, focused ultrasound therapy, cryotherapy, or any combination thereof. In certain embodiments, the method further comprises subjecting the cancer cell to radiation therapy, surgery, thermos-ablation, focused ultrasound therapy, cryotherapy, or any combination thereof.

When used in combination with cancer therapies, such as chemotherapies, the stapled peptide can enable a reduction in the amount of therapy/chemotherapy needed. The benefit to be realized is decreased toxicity, which may also result in improved outcomes.

The method can be adapted to treat conditions in which the level of NRF2 is elevated, such as when NRF2 is not degraded, degraded at an abnormally low level, constitutively activated, or otherwise over-expressed.

Uses of the stapled peptides and composition thereof are also provided. In certain embodiments, a use of a stapled peptide or composition hereof in the preparation of a medicament is provided. The medicament can be for the sensitization of a cancer to a cancer therapy. The cancer therapy can comprise administration of a chemotherapeutic agent to a subject. The chemotherapeutic agent can be cisplatin.

While the present disclosure has been made with reference to humans, it is contemplated that the disclosure has utility for other species, such as other species of mammals. Stapled peptides can be designed for a particular species and then used to treat members of that species in accordance with the teachings provided herein.

The terms “treat,” “treating,” “treated,” and “treatment” (with respect to a disease or condition, such as cancer) are used to describe a method for obtaining beneficial or desired results, such as clinical results, which can include, but are not limited to, one or more of improving a condition associated with a disease, curing a disease, lessening severity of a disease, increasing the quality of life of one suffering from a disease, prolonging survival and/or a prophylactic treatment. In reference to cancer, in particular, the terms “treat,” “treating,” “treated.” or “treatment” can additionally mean reducing the size of a tumor, completely or partially removing the tumor (e.g., a complete or partial response), stabilizing a disease, preventing progression of the cancer (e.g., progression-free survival), or any other effect on the cancer that would be considered by a physician to be a therapeutic or prophylactic treatment of the cancer. More particularly, curative treatment refers to any of the alleviation, amelioration and/or elimination, reduction and/or stabilization (e.g., failure to progress to more advanced stages) of a sign/symptom, as well as delay in progression of a sign/symptom of a particular disorder.

Prophylactic treatment refers to any of the following: halting the onset, reducing the risk of development, reducing the incidence, delaying the onset, reducing the development, and increasing the time to onset of symptoms of a particular disorder. Desirable effects of treatment can include, but are not limited to, preventing occurrence or recurrence of a disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compositions are used to delay development of a disease and/or tumor, or to slow (or even halt) the progression of a disease and/or tumor growth.

As used herein, the term “administering” and its grammatical variants include all means of introducing the stapled peptides and pharmaceutical compositions comprising same, to a subject. The term “subject” includes human and non-human animals, such as companion animals (dogs and cats and the like) and livestock animals. Livestock animals are animals raised for food production. Examples of administering include, but are not limited to, oral (po), parenteral, systemic/intravenous (iv), intramuscular (im), subcutaneous (sc), transdermal, intrasternal, intraarterial, intraperitoneal, epidural, intraurethral, intranasal, buccal, ocular, sublingual, vaginal, rectal, and the like.

Illustrative means of parenteral administration include needle (including microneedle) injectors, needle-free injectors and infusion techniques, as well as any other means of parenteral administration recognized in the art. Parenteral formulations are typically aqueous solutions, which may contain excipients, such as salts, carbohydrates and buffering agents (preferably at a pH in the range from about 3 to about 9). The preparation of parenteral formulations under sterile conditions may readily be accomplished using standard pharmaceutical techniques well-knovwn to those skilled in the art.

The stapled peptides can be formulated as pharmaceutical compositions and administered to a subject, such as a human patient, in a variety of forms adapted to the chosen route of administration. For example, the pharmaceutical composition can be formulated for and administered via oral or parenteral, intravenous, intraarterial, intraperitoneal, intrathecal, epidural, intracerebroventricular, intraurethral, intrasternal, intracranial, intratumoral, intramuscular, topical, inhalation and/or subcutaneous routes. Indeed, the stapled peptides and compositions hereof can be administered directly into the blood stream, into muscle, or into an internal organ.

The peptides and/or compositions can be administered via infusion or injection (e.g., using needle (including microneedle) injectors and/or needle-free injectors). Solutions of the composition can be aqueous, optionally mixed with a nontoxic surfactant and/or can contain carriers or excipients such as salts, carbohydrates and buffering agents (preferably at a pH of from 3 to 9).

The percentage of stapled peptides and/or compositions and preparations may vary and may be between about 1 to about 99% weight of the active ingredient(s) and a binder, excipients, a disintegrating agent, a lubricant, and/or a sweetening agent (as are known in the art). The amount of the stapled peptides in such therapeutically useful compositions is such that a cancer therapy-sensitizing dosage level will be obtained.

Depending upon the route of administration, a wide range of permissible dosages are contemplated herein. The dosages may be single or divided and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

Multiple infusions may be required in order to treat a subject effectively. For example, 2, 3, 4, 5, 6 or more separate infusions may be administered to a subject at intervals of from about 24 hours to about 48 hours, or every 3, 4, 5, 6, or 7 days. Infusions may be administered weekly, biweekly, or monthly. Monthly administrations can be repeated from 2-6 months or longer, such as 9 months to year.

### General

All patents, patent application publications, journal articles, textbooks, and other publications mentioned in the specification are indicative of the level of skill of those in the art to which the disclosure pertains and incorporated herein by reference in their entireties.

In the above description, numerous specific details are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary but remain applicable in view of the data provided herein.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components.

It should be noted that a connection between two components does not necessarily mean a direct, unimpeded connection, as a variety of other components may reside between the two components of note. Consequently, a connection does not necessarily mean a direct, unimpeded connection unless otherwise noted.

Further, will be understood that the disclosure is presented in this manner merely for explanatory purposes and the principles and embodiments described herein may be applied to compounds and/or composition components that have configurations other than as specifically described herein. Indeed, it is expressly contemplated that the components of the composition and compounds of the present disclosure may be tailored in furtherance of the desired application thereof.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein.

The term “about,” when referring to a number or a numerical value or range (including, for example, whole numbers, fractions, and percentages), means that the number or numerical range referred to is an approximation within experimental variability (or within statistical experimental error), and thus the numerical value or range can vary between 1% and 15% of the stated number or numerical range (e.g., +/−5% to 15% of the recited value), provided that one of ordinary skill in the art would consider equivalent to the recited value (e.g., having the same function or result).

When ranges are used herein for physical properties, such as molecular weight, or chemical properties, such as chemical formulae, all combinations and sub-combinations of ranges and specific embodiments therein are intended to be included.

The disclosure may be suitably practiced in the absence of any element(s) or limitation(s), which is/are not specifically disclosed herein. Thus, for example, each instance herein of any of the terms “comprising,” “consisting essentially of.” and “consisting of” (and related terms such as “comprise” or “comprises” or “having” or “including”) can be replaced with the other mentioned terms. Likewise, the singular forms “a,” “an,” and “the” include plural references unless the context clearly dictates otherwise. Thus, for example, references to “the method” include one or more methods and/or steps of the type, which are described and/or which will become apparent to those ordinarily skilled in the art upon reading the disclosure. The term “substantially” can allow for a degree of variability in a value or range, for example, within 90%, within 95%, or within 99% of a stated value or of a stated limit of a range.

It is recognized that various modifications are possible within the scope of the disclosure. Thus, although the present disclosure has been specifically disclosed in the context of preferred embodiments and optional features, those skilled in the art may resort to modifications and variations of the concepts disclosed herein. Such modifications and variations are considered to be within the scope of the disclosure as claimed herein.

It is therefore intended that this description and the appended claims will encompass all modifications and changes apparent to those of ordinary skill in the art based on this disclosure. For example, where a method of treatment or therapy comprises administering more than one treatment, compound, or composition to a subject, it will be understood that the order, timing, number, concentration, and volume of the administration is limited only by the medical requirements and limitations of the treatment (i.e., two treatments can be administered to the subject, e.g., simultaneously, consecutively, sequentially, alternatively, or according to any other regimen).

Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

Further, the use of headings and subheadings is for ease of reference, given the length of the document. Description under one heading or subheading (such as a subheading in the Detailed Description) is not intended to be limited to only the subject matter set forth under that particular heading or subheading.

### EXAMPLES

The following examples serve to illustrate the present disclosure. The examples are not intended to limit the scope of the claimed invention in any way.

### Materials and Methods

General methodologies and materials used in the Examples described herein are provided in this section. Any study-specific modifications to these general materials and methods will be set forth in the particular Example.

Instrumentation: A PS3 peptide synthesizer (Gyros Protein Technologies AB, Tucson, AZ) or a liberty blue 2.0 microwave peptide synthesizer (CEM Corporation, Charlotte, NC) was used for peptide synthesis. An Agilent Technologies 1260 Infinity II preparative high-performance liquid chromatography (HPLC) system was used for peptide purification. An Advion Expression CMS single quadrupole mass spectrometer and an Rda Waters UPLC-mass spectrometer were used for peptide confirmation. The Jasco J-1500 Circular Dichroism Spectrophotometer was used for circular dichroism experiments, and the Nikon A1 Confocal was used for imaging fluorescent peptides for cell permeability assays.

Reagents: Fmoc-protected α-amino acids (other than the olefinic amino acid Fmoc-(S)-2-amino-2-methyl-hept-6-enoic acid (Fmoc-S5-OH), which was purchased from Sigma-Aldrich (St. Louis, MO)) were purchased from Chem-Impex International (Wood Dale, IL). Rink amide MBHA resin was purchased from Sigma-Aldrich (St. Louis, MO). Dimethylfonnamide (DMF), N,N-diisopropylethylamine (DIEA), acetic anhydride, trifluoroacetic acid (TFA), dichloromethane (DCM), anisole, 1, 2-dichloroethane (DCE), triisopropylsilane (TIPS), fluorescein isothiocyanate isomer 1 (FITC), Biotin-Osu and piperidine were purchased from Millipore Sigma (Burlingon, MA) and used as supplied. The Grubbs catalyst first-generation catalyst was obtained from Sigma-Aldrich (St. Louis, MO).

Cell lines: The cell lines A549 and Hek293T were purchased from American Type Culture Collection (ATCC). Hek293T cells were cultured in Dulbecco's Modified Eagle Medium (DMEM) supplemented with 10% fetal calf serum, stable L-glutamine and 1× non-essential amino acids. A549 cells were maintained in RPMI-1640 supplemented with 10% fetal calf serum and L-glutamine. Cultures were maintained at 37 °C in a humidified atmosphere of 5% CO₂.

### Example 1

### Design and Synthesis of Stapled Peptide NRF2 Inhibitors

Stapled peptides based on NRF2 sequences were designed with the goal of identifying direct inhibitors of NRF2-sMAF heterodimerization. Focus was placed on Nrf2 peptides, rather than MafG peptides, because Nrf2 is known to be unstructured when not bound to either KEAP1 or the sMAFs.

Primarily, the Nrf2 sequence was obtained via polymerase chain reaction (PCR) from the human cDNA library. The insert was cloned into pMalc2 with Barn 1-H and Xba 1. After transformation into Rosetta (DE3), the cells were grown in 2× yeast extract tryptone (2XYT) media to OD=0.3 at 37 °C and protein expression was induced by isopropyl β-d-1-thiogalactopyranoside (IPTG) (1 mM) at 37 °C for 2.5 hours. After collection of the cell pellet by centrifugation, the pellet was lysed by a microfluidizer in 1× carbonate-bicarbonate (CB) buffer with 2% Triton X-100, dithiothreitol (DTI) 1 mM, and phenylmethylsulfonyl fluoride (PMSF) 1 mM. The target protein was incubated in and bound to amylose resin, then washed with CB buffer (10 mM Tris buffer with 200 mM NaCl, pH7.4). The target protein was eluted by 10 mM maltose in 1× CB buffer. The eluted protein was dialyzed against 1× CB buffer with 20% glycerol and then stocked at −80 °C.

Similarly, the MafG sequence was obtained via PCR from the human cDNA library. The insert was cloned into pET15B with Nde I and Ban HI. After transformation into Rosetta (DE3), the cells were grown in 2XYT media to OD=0.3 at 37 °C, and then protein expression was induced by IPTG 1 mM at 37 °C for 2.5 hours. After collection of the cell pellet by centrifugation, the pellet was lysed by a microfluidizer in 1× CB buffer with 2% Triton X-100, DTT 1 mM, and PMSF 1 mM. The target protein was incubated in and bound to Ni-NTA resin, then washed with 50 mM imidazole in CB buffer. The target protein was eluted with 250 mM imidazole in CB buffer. The eluted protein was dialyzed against 1× CB buffer with 20% glycerol and then stocked at −80 °C.

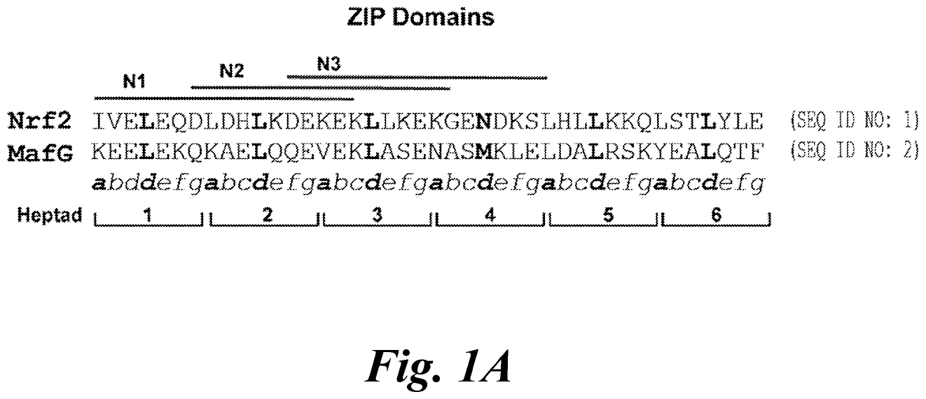

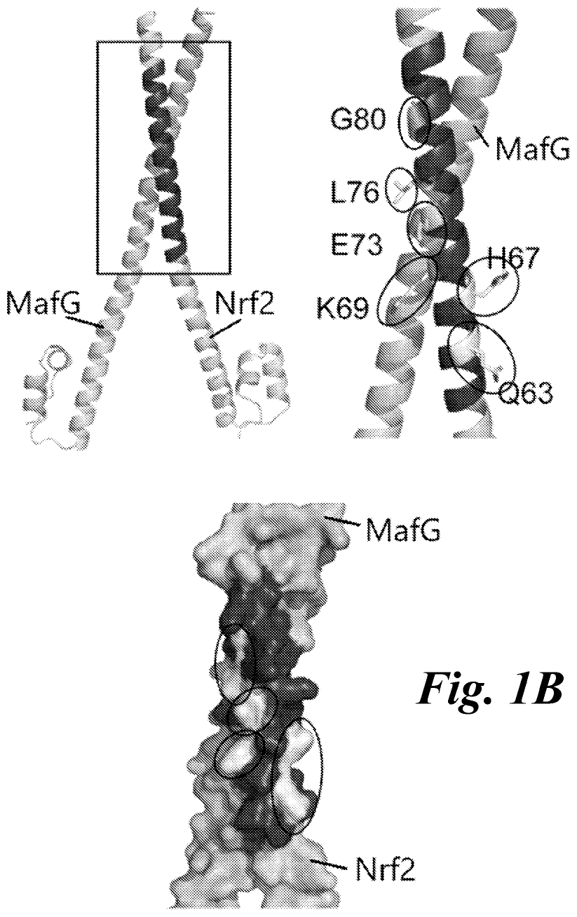

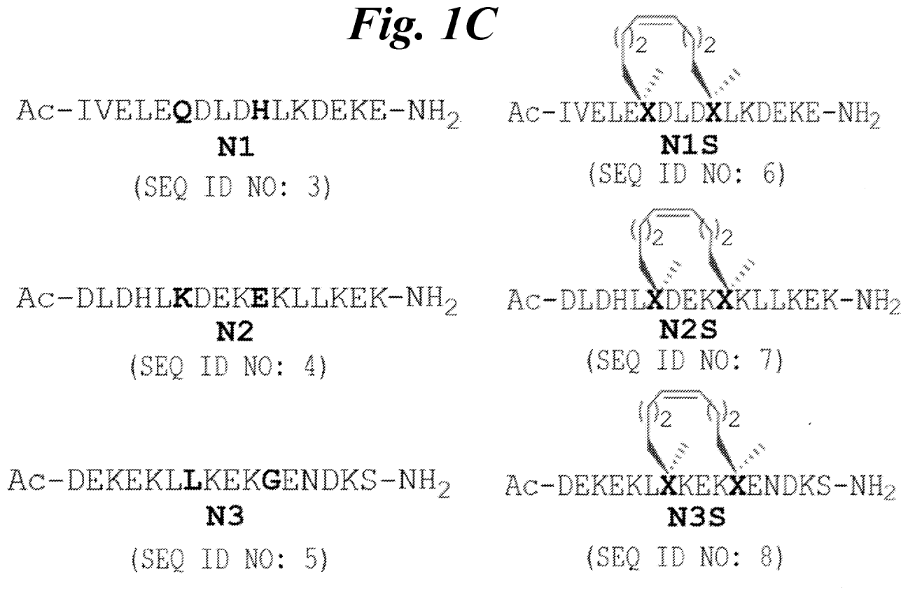

AlphaFold was used to predict the structure of the Nrf2/MafG heterodimer (FIG. 1B). Briefly, protein models were generated using Alphafold2 and Alphafold2-multimer. Sequence alignments were generated using Mmseq2 and Hhsearch. Dinkova-Kostova et al., Direct Evidence That Sulfhydryl Groups of Keap1 Are the Sensors Regulating Induction of Phase 2 Enzymes That Protect against Carcinogens and Oxidants. PNAS 99(18): 11908-11913 (2002). Query sequences were submitted to the server using colabFold, allowing for quick alignment and modeling. Mirdita et al., ColabFold: Making protein folding accessible to all, Nature Methods 19(6): 679-682 (2022).

The structure predicted by AlphaFold agreed well with previous studies that demonstrated experimentally the necessity of the ZIP domains for interaction of Nrf2 with sMAFs such as MafG. Itoh et al., An Nrf2/Small Maf heterodimer mediates the induction of phase II detoxifying enzyme genes through antioxidant response elements, Biochemical & Biophysical Research Communications 236(2): 313-322 (1997).

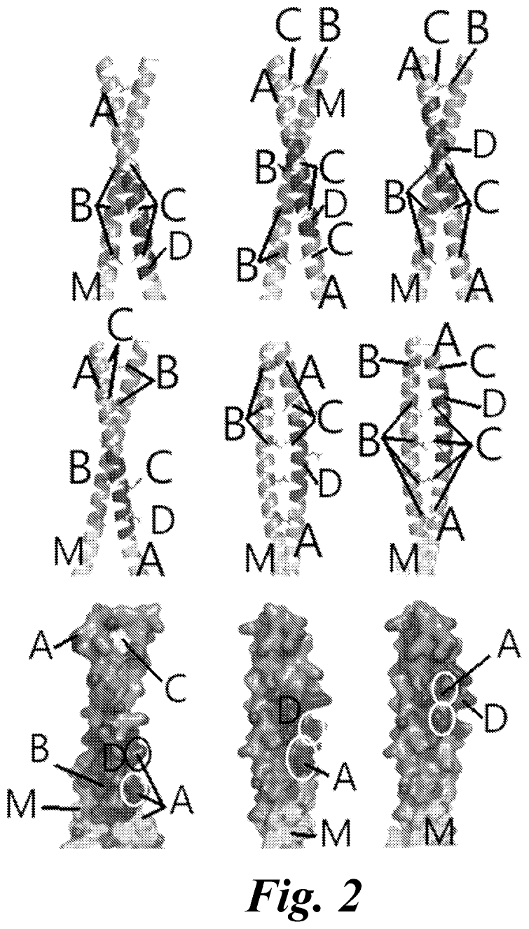

The AlphaFold model was used to determine the best region to target for inhibition. Specifically, three 16-amino acid peptides were designed to cover the predicted binding domain. The length of the peptides (16 amino acids) was based on other cell-permeable stapled peptides. Chang et al., Stapled α-helical peptide drug development: a potent dual inhibitor of MDM2 and MDMX for P53-dependent cancer therapy, Proceedings Nat'l Academy Sciences (PNAS) USA 110(36): E3445-E3454 (2013); Migon et al., Hydrocarbon stapled antimicrobial peptides, Protein J 37(1): 2-12 (2018); Ali et al., Stapled peptides inhibitors: a new window for target drug discovery, Computational & Structural Biotechnology J 17: 263-281 (2019); Klein. Stabilized helical peptides: overview of the technologies and its impact on drug discovery, Expert Opinion on Drug Discovery 12(11): 1117-1125 (2017); Li et al., Stapled helical peptides bearing different anchoring residues, Chemical Reviews 120(18): 10079-10144 (2020). The model also was used to determine the best locations for staples, specifically focusing on locations unlikely to interrupt binding of the Nrf2 peptides to MafG (FIGS. 1B-1C and FIG. 2).

Stapled peptides (compounds NIS, N2S, and N3S) and unstapled peptides (compounds N1, N2, and N3) were synthesized using standard Fmoc-based, solid phase peptide synthesis. Further, Grubbs metathesis was followed for stapled peptides. FIG. 1C shows each of the stapled and unstapled compounds.

Briefly, for resin loading for linear peptide synthesis, rink amide MBHA resin (1 g, 0.68 mmol g-1, 0.68 mmol, 100-200 mesh) was swelled in dimethylformanide (DMF) for 30 minutes, drained, and treated with a solution of Fmoc-protected amino acid (2 nmmol) and N, N′-dicyclohexylcarbodiimide (1 mmol) in DMF (11 mL). The resulting mixture was gently agitated for 2 hours, after which the resin was filtered and washed with DMF (2×5 mL). Remaining unreacted amide groups were capped by agitating the resin with acetic anhydride and DIEA in 4.5 ml DMF (0.5:0.5:9) for 20 minutes. The resin was filtered and washed with dichloromethane (DCM) (3×5 mL) and MeOH (3×5 mL) and dried under vacuum for 1 hour. The resin loading was determined by treating an aliquot (1-3 mg) of the dried resin with piperidine-DMF (1:4) and observing the UV absorbance of the piperidine-dibenzofulvene adduct at 301 nm (ε=7800 M⁻¹ cm⁻¹). Hostetler et al., Synthetic natural product inspired cyclic peptides. ACS Chemical Biology 16(11): 2604-2611 (2021).

For automated linear peptide synthesis, pre-loaded rink amide resin was used for all synthesis, with synthesis performed as described above. Id.

For automated stapled peptide synthesis, Liberty blue 2.0 (CEM Corporation, Charlotte, NC) was used for making the linear precursor along with performing ring-closing metathesis on the peptide while still on the rink amide solid support. For a 0.05 M scale, 0.2 M amino acids, 0.5 M DIC, 0.5 Oxyma and 20% piperidine in DMF were used for synthesis. After the amino acids were assembled, the N-terminal amine was acetylated using a 10% solution of acetic anhydride in DMF. The peptide resin was cyclized in the presence of 10 mM Grubbs catalyst in 1,2-dichloroethane. A ring-closing metathesis (RCM) reaction was verified by cleaving a small portion (~1 mg) of the peptide from the resin and performing liquid chromatography-mass spectrometry (LCMS). The resin-bound peptide was washed three times with DMF and three times with DCM and dried under vacuum.

Thereafter, resin was swelled in DMF for 5 minutes followed by Fmoc deprotection with 20% piperidine in DMF for 1 minute at 90 °C. The first Fmoc amino acid was loaded on the resin with N,N′-Diisopropylcarbodiimide (DIC) (0.5 M) in DMF and Oxyma (0.5 M) in DMF, reacted for 2 minutes at 90 °C followed by Fmoc deprotection with 20% piperidine in DMF, and reacted for 1 minute at 90 °C. The following Fmoc-protected amino acids (0.2 M) were coupled using DIC (0.5 M) in DMF and Oxyma (0.5 M) in DMF, and deprotected with 20% piperidine in DMF following the same reactional conditions. Following final Fmoc deprotection, the N-terminus was acetylated using 20% Ac₂O in DMF and reacted for 2 minutes at 60 °C. All Fmoc-Arg-OH and amino acids coupled following the stapling residues Fmoc-(S)-2-(4-pentenyl)alanine-OH were double-coupled (0.2 M×2) at 90 °C. RCM was performed using Grubb's I catalyst (10 mM) in DCE, reacting for 5 minutes at 90 °C. This was carried out twice. DMF and DCM washes were performed following coupling, deprotection, acetylation, and RCM steps. Paquette et al., RpoN-based stapled peptides with improved DNA binding suppress Pseudomonas aeruginosa virulence, RSC Medicinal Chemistry 13(4): 445-455 (2022).

Fmoc-Ahx-OH was coupled as a spacer to peptides subjected to fluorescein isothiocyanate (FITC) or biotin modification on the N-terminus, instead of acetyl capping. FITC-labelling was performed with a 4-fold excess of FITC and 8 eq. N,N′-Diisopropylethylamine (DIPEA) in (3:1) DMF/DCM overnight. Biotin was coupled using 2 eq. Biotin-Osu and 8 eq DIPEA in (3:1) DMF/DCM overnight.

Peptides were deprotected and cleaved from the solid support by treatment with trifluoroacetic acid (TFA)/H₂O/triisopropylsilane (TIS) (95/2.5/2.5 vol/vol) for 2 hours at room temperature. After filtration of the resin, the TFA solution was evaporated, and peptide was precipitated by addition of cold diethyl methyl ether mixture and centrifuged, frozen, and lyophilized to yield the desired product as a solid. Hostetler et al. (2021), supra.

HPLC analysis and purification were performed on an Agilent Technologies 1260 Infinity II preparative HPLC system using a 1260 variable wavelength detector (measuring at 214 nm and 254 nm). A Luna C18 reverse phase 5 μm, 150×4.6 mm column (Phenomenex, Torrance, CA) was used for purity analysis, and a Luna C18 reverse phase, 5 m, 150×21.2 mm column (Phenomenex, Torrance, CA) was used for purification as previously described. Hostetler et al. (2021), supra.

Mass spectra (MS) were recorded on an Advion Expression CMS single quadrupole mass spectrometer using electrospray ionization (ESI Group, Rungis, France) or a Waters ultra-performance liquid chromatography (UPLC) attached to an Rda detector.

The alpha helicity of the stapled (compounds N1S, N2S. and N3S) and unstapled peptides (compounds N1, N2, and N3) was then evaluated by using circular dichroism (CD) spectroscopy obtained on the Jasco J-1500 Circular Dichroism Spectrophotometer using measurement parameters of temperature 37 °C, wavelength 190-260 nm, step resolution 0.5 nm, speed 20 nm/s, accumulations 3, response 1 s, bandwidth 1 nm, and path length 0.2 cm. Each peptide was dissolved in deionized water at a concentration of 100 μM. The percentage helicity was calculated from the absorbance at 222 nm using helical models as previously reported. Chen, Determination of the helix and p form of proteins in aqueous solution by circular dichroism, Biochemistry 13(16): 3350-3359 (1974).

Peptide stability assays were performed using the protocols described in Cromm et al., Protease-resistant and cell-permeable double-stapled peptides targeting the Rab8a GTPase, ACS Chemical Biology 11(8): 2375-2382 (2016). Briefly, 55 μM of peptide was incubated with 10 μg/mL proteinase K for the indicated timepoints. At the end of each time point, 50 μL of the reaction mixture were quenched in 100 μL of a 1:1 mixture acetonitrile/water+1% formic acid (FA). Samples were injected and analyzed on a Triple quad. Analysis was done using an Agilent Technologies 1200 Series attached to an Agilent Technologies 6460 Triple Quad LC/MS. Analysis was performed with a ZORBAX XDB-C18 3.5 mm, 2.1×50 mm column (Agilent Technologies, Inc., Santa Clara, CA). Eluent A: H₂O with 0.1% FA. Eluent B: MeCN with 0.1% FA. Purity analysis utilized the gradient as follows: (A:B, 0.3 mL/min): 95:5, 0 minutes; 95:5, 1 minute; 5:95, 5 minutes; 5:95, 8 minutes: 95:5, 8.5 minutes; 95:5, 12.5 minutes.

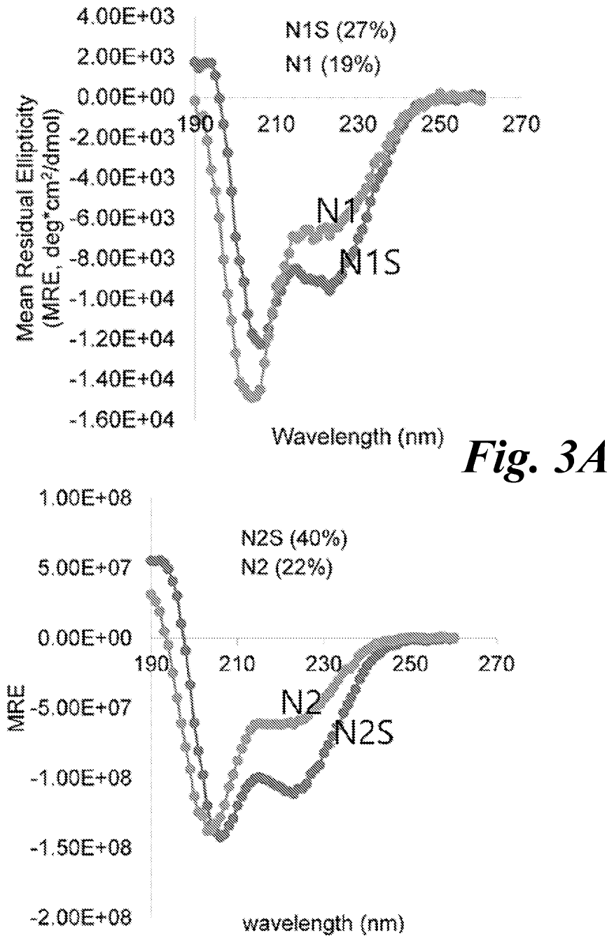

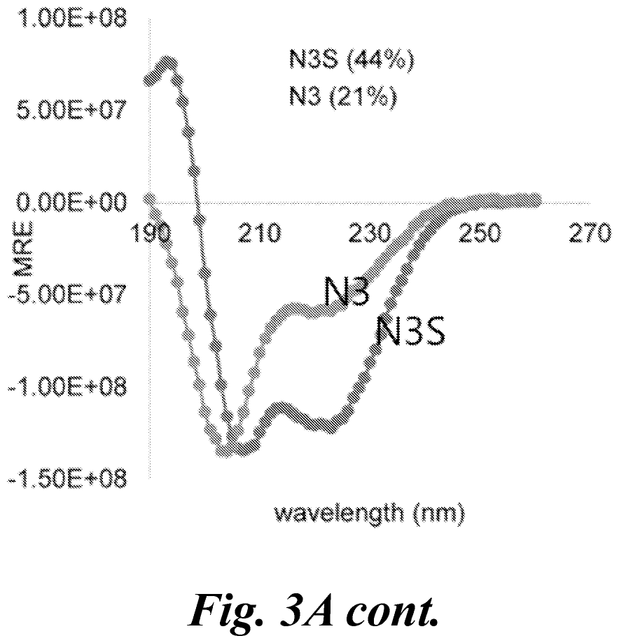

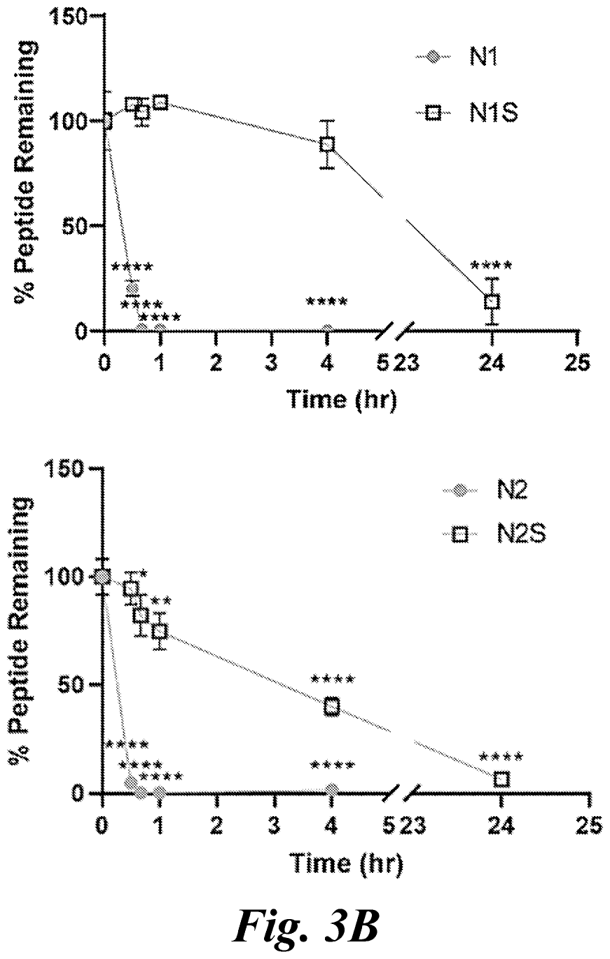

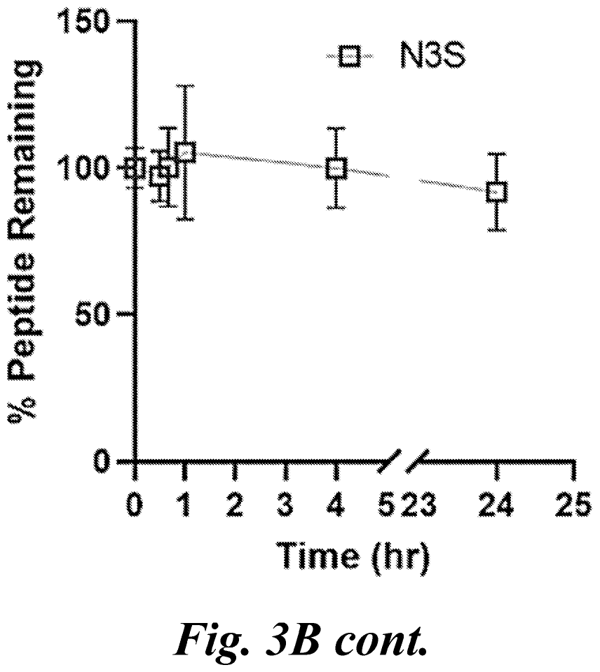

Circular dichroism measurements indicated that all stapled peptides had higher α-helicity compared to their corresponding unstapled peptides (FIG. 3A). Additionally, they had greatly increased protease stability compared to their unstapled versions (FIG. 3B). N3S showed no significant degradation for up to 24 hours. NIS showed some significant degradation starting at 24 hours, while N2S showed significant degradation starting at 40 minutes.

### Example 2

### Screening Peptides and Derivatives for Activity

The peptides from Example 1 were screened for Nrf2 inhibition using a previously developed dual luciferase assay. Ren et al., Brusatol enhances the efficacy of chemotherapy by inhibiting the Nrf2-mediated defense mechanism, Biological Sciences 108(4): 1433-1438 (2011). Briefly, HEK-293T cells stably expressing ARE-firefly luciferase and TK-Renilla luciferase were used. The cells were treated with different concentrations of compounds N1 (SEQ ID NO: 3), N1 S (SEQ ID NO: 6), N2 (SEQ ID NO: 4), N2S (SEQ ID NO: 7), N3 (SEQ ID NO: 5), and N3S (SEQ ID NO: 8) for 16 hours. The cells were lysed in 1× passive lysis buffer, and both firefly luciferase and Renilla luciferase activities were measured using the Promega Dual-Luciferase™ Reporter (DLR™) Assay System and a luminometer (Turner Biosystems 20/20ⁿ Luminometer). The samples were in triplicate for all concentrations of compounds (n=3).

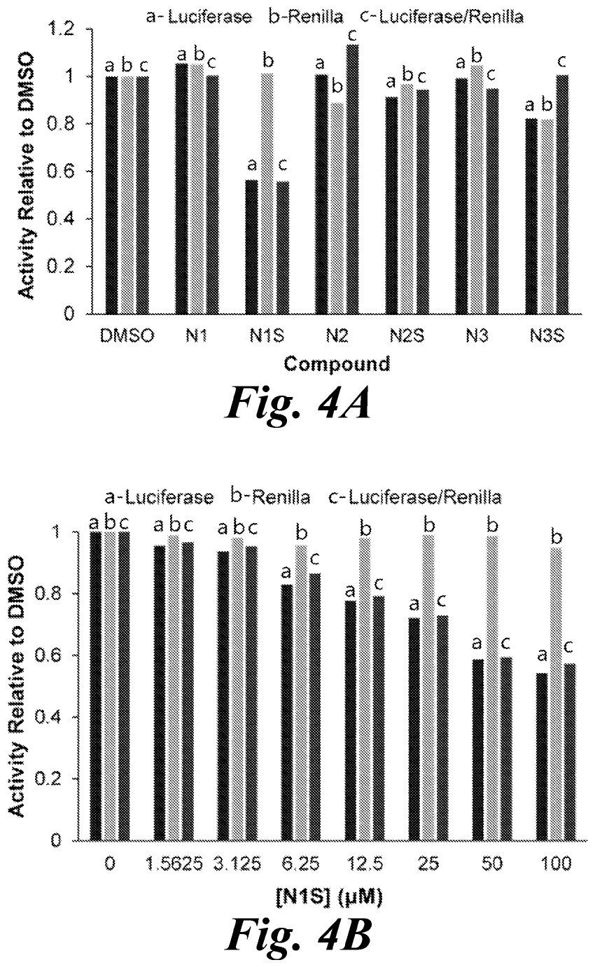

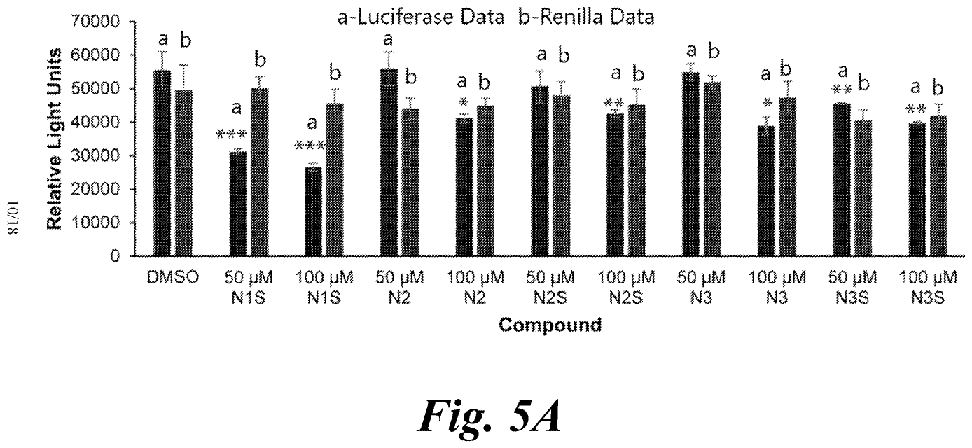

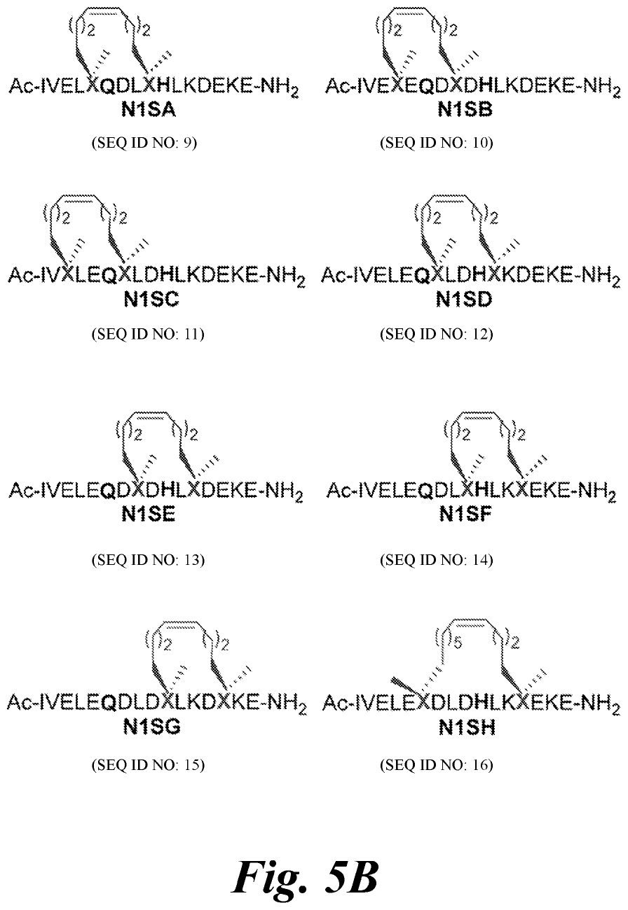

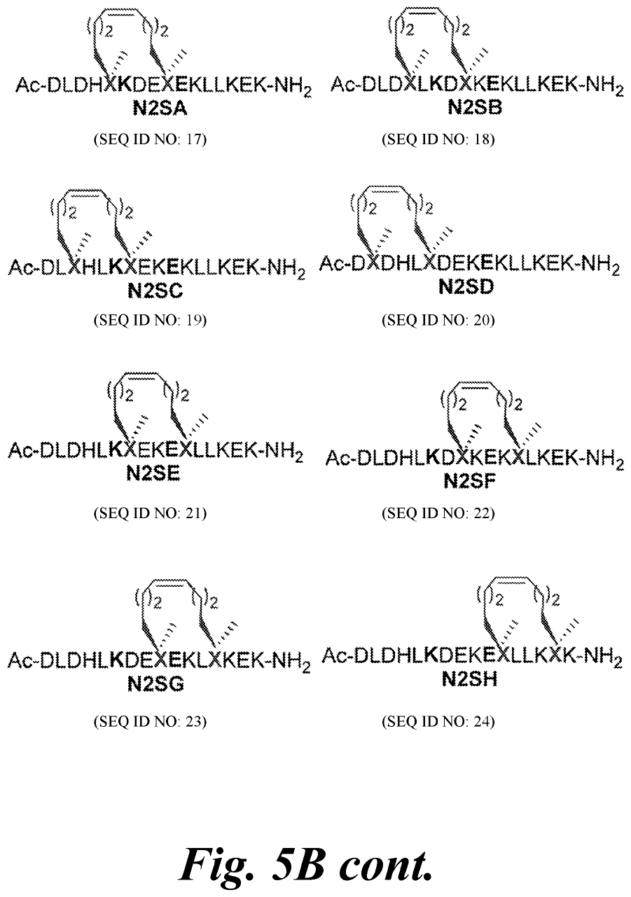

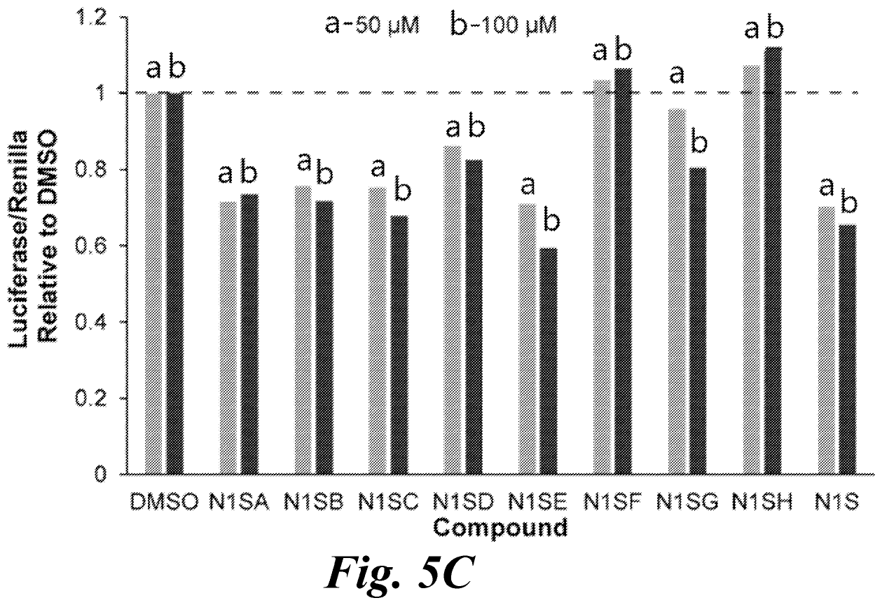

Of all the peptides tested, compound NIS (SEQ ID NO: 6) showed the most potent inhibition of Nrf2 activity while showing no toxicity to the cells (FIGS. 4A and 5A). Compounds N2, N2S, N3, and N3S all showed similar levels of activity. To determine whether this was a result of poor cell permeability by compounds N2S and N3S, fluorophore labeled versions of these peptides were generated, and confocal microscopy was used to determine the abilities of these peptides to enter cells (FIG. 5C).

Briefly, A549 cells at 9.5×10⁴ cells/ml in 600 μl media were plated on 10 mm glass-bottomed, 24-well plates (Chemglass Life Sciences Cell Culture Plate, 24-Well, Fisher Scientific, Waltham, MA) and cultured for overnight at 37 °C, 5% CO₂, in complete growth medium (RPMI supplemented with 10% fetal calf serum, L-glutamine, and non-essential amino acids). The next day, cells were treated with fresh, pre-warmed complete growth medium supplemented with the desired FITC-labeled peptide or DMSO vehicle control at a final concentration of 100 μM FITC labeled peptide and 1% DMSO. After 90 minutes of treatment, cells were washed three times in warm PBS to remove excess peptide from the cell surface and left in modified Krebs-Ringer buffer solution [KRBH: 134 mM NaCl. 3.5 mM KCl. 1.2 mM KH₂PO4, 0.5 mM MgSO4, 1.5 mM CaCl₂), 5 mM NaHCO₃, 10 mM HEPES (pH 7.4)] containing Hoechst stain at 1 μg/ml. Cells were immediately analyzed under a Nikon A1 confocal microscope (Resonant). The images were taken at 20× magnification with fluorescence excitation wavelengths at 409 for the Hoechst and 488 for the FITC. Images were processed using NIS-Elements AR. Dietrich et al., Cell permeable stapled peptide inhibitor of Wnt signaling that targets 0-catenin protein-protein interactions, Cell Chemical Biology 24(8): 958-968.e5 (2017); Dougherty et al., Enhancing the cell-permeability of stapled peptides with a cyclic cell-penetrating peptide, J Medicinal Chemistry 62(22): 10098 (2019).

Compounds NIS and N2S showed good cellular and nuclear permeability. Compound N3S showed minimal cellular permeability with very little to no accumulation in the nucleus. Compound N1 showed poor cellular permeability. This suggests the lack of compound N2S and compound N3S activity in the luciferase assays was most likely a result of poor peptide stability and peptide permeability, respectively.

Because compound NIS showed the most promise, it was evaluated for dose response (FIG. 4B). It showed a dose-response curve for Nrf2 inhibition without showing toxicity. Sixteen derivatives of compound NIS and compound N2S with varying staple positions were examined. None of the compound N2S derivatives showed activity, suggesting that that scaffold is not a promising one. Several of the compound NIS derivatives showed activity, but none had more activity than compound N1S (FIGS. 5B and 5C). Therefore, the mechanism of compound N1S was examined.

### Example 3

### In Vitro Activity

To determine whether compound N1S directly inhibits the NRF2-sMAF-ARE association, a fluorescence polarization assay with Nrf2, MafG, and fluorophore-labeled ARE was performed. Briefly, a 96-well plate (VWR® Solid Bottom Assay Plates, 96-well, black) was used to encapsulate each fluorescence polarization reaction. Unless otherwise stated (i.e., ARE only, ARE+MafG), each 100-μL reaction contained 200 nM purified recombinant His-MBP-NRF2, 200 nM purified recombinant His-MafG, various concentrations of molecule, and 20 nM 5′ FAM-labeled ARE all together in 1× PBS solution.

First, NRF2 was incubated with molecule for 5 minutes with shaking, followed by the addition of MafG for another 5-minute incubation. Finally, ARE was added, and the MafG-NRF2-ARE system was incubated for 10 minutes with shaking. After incubation, the plate was placed into a plate reader (Molecular Devices SpectraMax® iD5 Multi-Mode Microplate Reader). The fluorescence polarization measurement was performed at excitation/emission values of 470:525 nm, respectively. The samples were in triplicate (n=3).

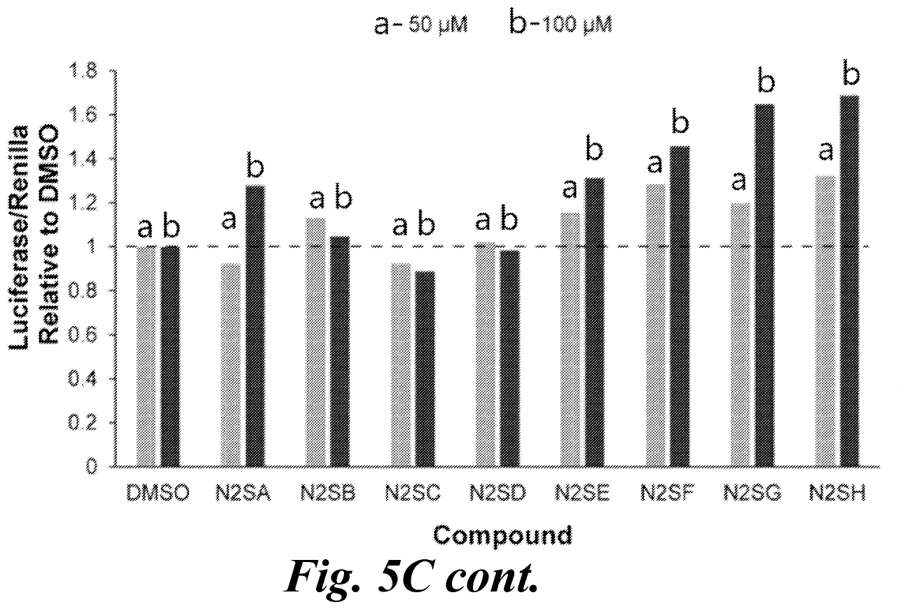

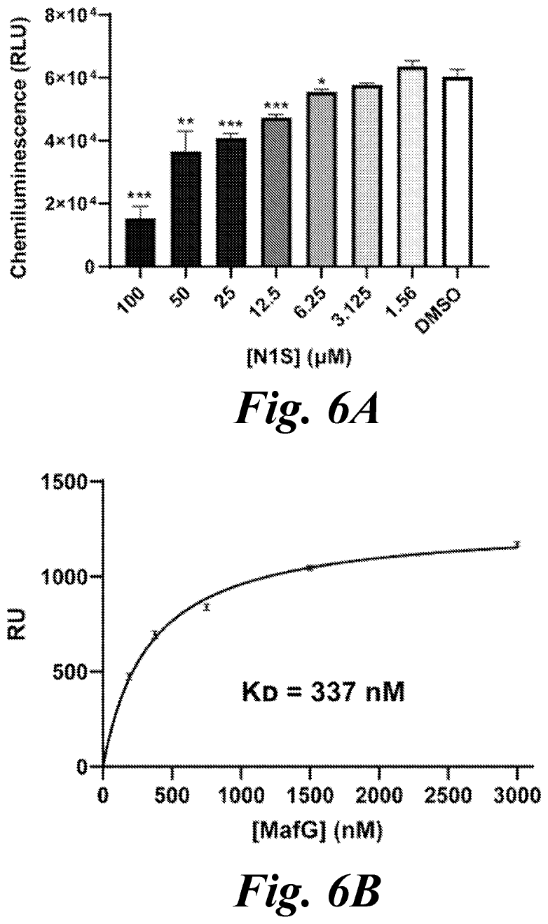

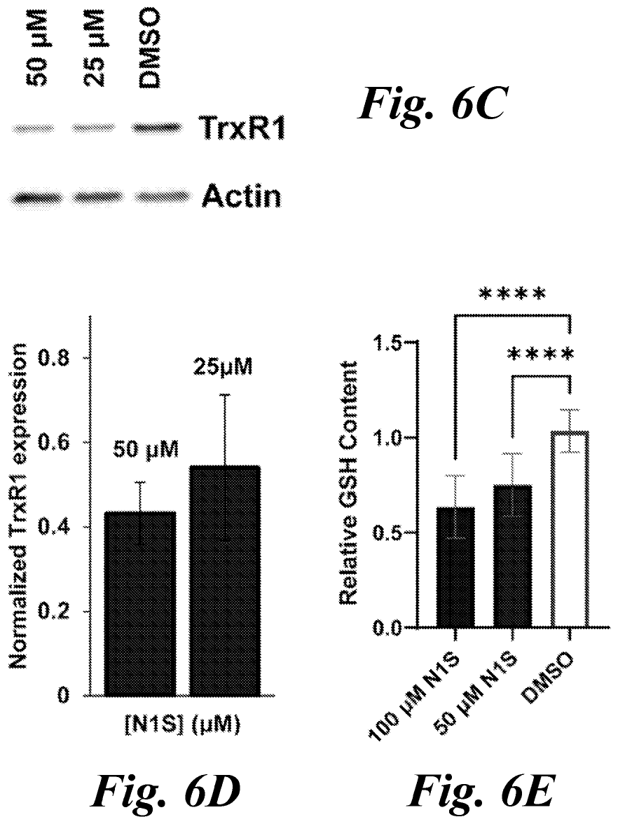

Compound N1S decreased the binding of the heterodimer to the DNA at concentrations as low as 0.2 M, supporting that the decrease in luciferase signal seen in the luciferase assay is due to direct disruption of the Nrf2-MafG-DNA ARE complex. Based on the design, it was expected that compound NIS directly inhibited heterodimerization of Nrf2 with sMAFs including MafG. The split-luciferase assay confirmed this, with compound NIS inhibiting dimerization of Nrf2 with MagF in vitro in a dose-dependent manner (FIG. 6A).

To show direct interaction between compound NIS and MafG, an immunoprecipitation assay was carried out with His-tagged, purified MafG and biotin-labeled compound NTS peptide. Briefly, a Pierce™ Biotinylated Protein Interaction Pull-Down Kit was purchased from Thermo Fisher, and manufacturer's instructions were followed. Purified His-tagged MafG and biotin-tagged NIS were used as prey proteins. For competition experiments, 20× unlabeled NIS was added after 1 hour incubation with MafG. Compound N1S-biotin successfully interacted with MafG, and this interaction could be competed away by adding 20-fold unlabeled compound N1S peptide, providing strong evidence that it is specifically binding to the compound NIS peptide.

To determine the affinity of binding between MafG and compound NIS, surface plasmon resonance (SPR) was performed with biotin-labeled compound NIS and His-MafG. For all SPR-binding measurements, a Biacore 100-Instrument (Biacore-GE) was used. Binding between MafG and biotin-labeled compound NIS peptide was performed by first immobilizing the biotin N1S peptide on a SA chip (Cytiva, Marlborough, MA). The binding buffer (150 mM NaCl, 10 mM HEPES, pH 7.5, 1 mM MgCl2, 1 mM tris(2-chloroethyl)phosphate (TCEP) and 0.05% P-20) was used. Biotin-compound NIS (1 μM) was injected at a rate of 10 μl min⁻¹ for 10 minutes, immobilizing approximately 528 response units. Increasing concentrations of MafG (two-fold dilutions between 2.5 μM and 0.100 μM, including two blanks) were subsequently passed over the Biotin-compound N1S functionalized chip as well as a streptavidin reference surface. Multiple cycle analysis was carried out, without regenerating the chip between different MafG concentrations. Binding data was reference normalized and processed using Prism software, A one-site binding model was applied to the processed data set to determine binding parameters of MafG binding to N1S.

Using a one-site binding model, the dissociation constant was found to be 337 nM (FIG. 6B). Collectively, these data highly suggest that compound N1S can directly inhibit Nrf2/sMAF heterodimerization by directly binding the sMAF proteins.

After confirming the in vitro activity of NIS, its mechanism in a cellular context was examined. Western blots were carried out in the A549 non-small cell lung cancer cell line to show the effects of N1S inhibition on the protein expression of Nrf2-regulated proteins. Briefly, A549 cells were seeded in 6-well plates at a concentration of 2×10⁵ cells/ml in 2.5 ml. Cells were treated the next day with either peptide at the desired concentration or DMSO. After a 72-hour incubation, cells were collected by trypsinization and centrifugation. Pellets were washed with PBS three times before the pellet was resuspended in 250 μL of RIPA buffer containing protease inhibitor and kept on ice for 30 minutes with intermittent vortex. The cells were then spun at 16,000 g for 5 minutes, and the supernatant was collected. Protein concentration of lysate was normalized with Pierce BCA Protein Assay Kit (Thermo Fisher Scientific, Waltham, MA). Equal volumes of lysate were mixed with 2× Laemmli sample buffer before separating on 4-15% Mini Protean TGX SDS-PAGE (Bio-Rad Laboratories, Hercules, CA).

Protein content was then transferred to polyvinylidene fluoride (PVDF) membranes and blocked for 1 hour with a 5% nonfat dry milk in tris-buffered saline with 0.1% Tween® 20 detergent (TBST) solution. Membranes were incubated with primary antibodies overnight at 4 °C and washed three times for 5 minutes each with TBST. Horseradish peroxidase (HRP)-conjugated secondary antibodies were then added for 2 hours at room temperature. Membranes were washed an additional three times for 5 minutes each before visualizing on ChemiDoc (Bio-Rad Laboratories. Hercules, CA) SuperSignal West Pico Plus chemiluminescent substrate (Thermo Fisher Scientific. Waltham, MA).

ImageJ was used for quantification of all bands. Primary antibodies: TrxR1 (Santa Cruz Biotechnology, Dallas, TX), sc-28321, 1:100 dilution (~55 kDa band analyzed); j-actin (Cell Signaling Technology, Inc., Danvers, MA), #3700, 1:1000. Secondary antibodies: mouse IgG Fc binding protein (m-IgG Fc BP) conjugated to horseradish peroxidase (HRP), Santa Cruz Biotechnology, 525409, 1:500 used with TrxR1 primary antibody and anti-mouse IgG, #7076, Cell Signaling Technology, 1:5000 was used with j-actin primary antibody. Bollong et al., A small molecule inhibits deregulated NRF2 transcriptional activity in cancer, ACS Chemical Biology 10(10): 2193-2198 (2015). In A549 cells treated with N1S for 72 hours, a decrease in thioredoxin reductase 1 (TrxR1), a direct target of Nrf2, was observed (FIGS. 6C and 6D).

To ensure the biological relevance of these results, glutathione (GSH) levels in NTS-treated cells were quantified using a GSH-Glo™ Glutathione Assay (Promega Corporation, Madison, WI). The manufacturer's instructions were followed. Cells were plated at a density of 20,000 cells/ml and treated with various concentrations of NIS for 24 hours. Significant reductions at 24 hours were observed (FIG. 6E).

Overall, these data strongly support that compound NIS directly inhibits Nrf2/MafG interaction and their downstream activation of transcription of their gene targets.

### Example 4

### Sensitization of Cancer Cells to Chemotherapeutics

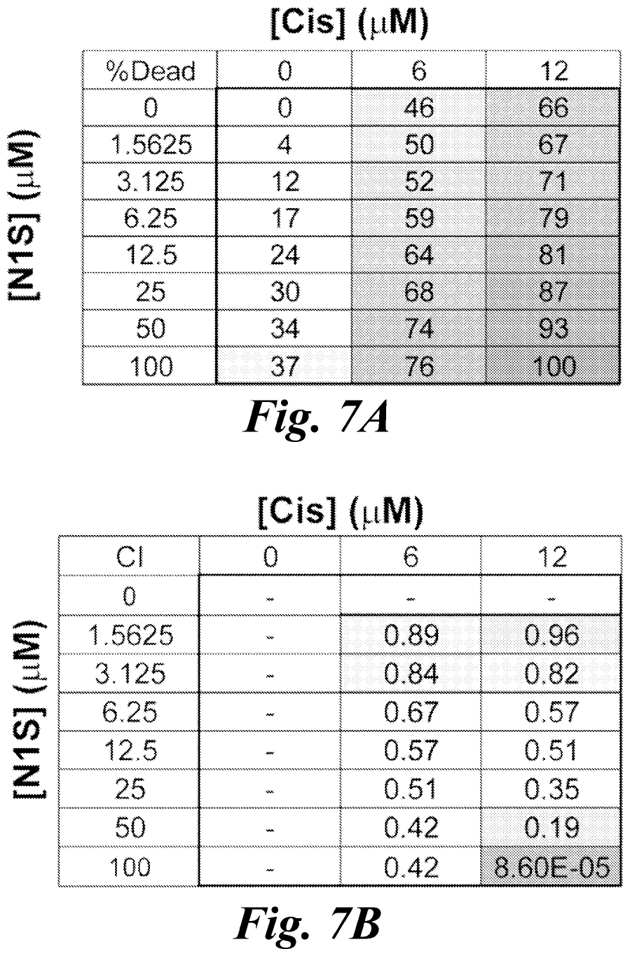

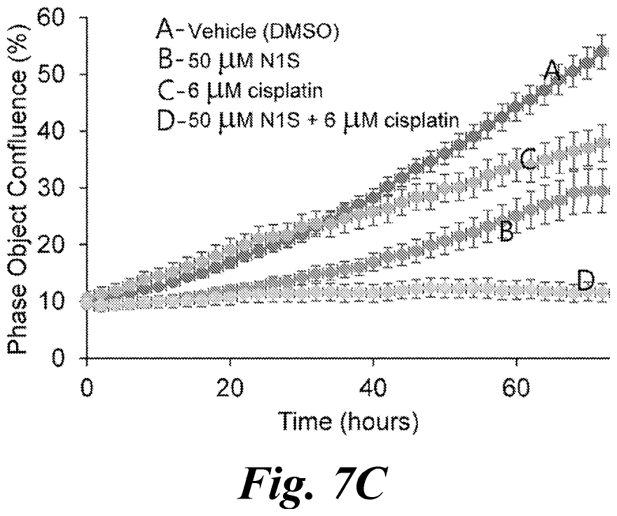

The ability of compound N1S to increase sensitivity to chemotherapeutics was examined. A549 non-small cell lung cancer cells were seeded into a Corning® 96-Well Cell Culture Plate (clear, sterile, non-treated) and incubated for 24 hours. The cells were treated with cisplatin alone, compound NIS alone, or in combination. Immediately following treatment, the cells were placed into an Incucyte® S3 Live-Cell Analysis Instrument (Sartorius AG, Gottingen, Germany) and imaged every two hours for three days. The Incucyte® S3's analytical software was used to calculate cell confluency at each time point. Synergy was determined using the Chou-Talalay method. The datapoints suggest that synergy was observed with relatively low concentrations of cisplatin and varying concentrations of N1S (FIG. 7C).

Additionally, an MTT assay was performed on the plate of cells from the Incucyte® study, and a separate, identical plate of cells was incubated for 24 hours. MTT reagent (20 μL of 5 mM stock) was added to each well and incubated for three hours at 37 °C. Following incubation, the plate was spun down at 2,500 rpm for five minutes at room temperature and then aspirated to remove the media. Isopropanol/HCl (100 μL of 53 μL concentrated HCl in 50 mL isopropanol) was added into each well, and the plate was placed on a shaker at room temperature for 10 minutes. The absorbance of each well was measured at 570 nm using a plate reader (Molecular Devices SpectraMax® iD5 Multi-Mode Microplate Reader). Each treatment group was plated in triplicate (n=3) (FIGS. 7A and 7C). These live cell analysis data further confirmed the Incucyte® study results, showing similar results of the combination of 6 μM cisplatin with 50 μM compound N1S and greatly reduced levels of growth compared to either treatment alone (FIG. 7B).

Finally, a hemolysis assay was performed based on a method described in Hostetler et al. (2021), supra. The peptides were tested at a final concentration of 100 μM and an average of 0.3% hemolysis was observed (data not shown).

Overall, these results demonstrate that NIS can greatly sensitize cancer cell lines to chemotherapeutics. NIS also showed no toxicity to red blood cells (0.3%±1% hemolysis at 100 μM), providing further support for it as a lead inhibitor to sensitize Nrf2-dependent cancers to chemotherapeutics.

1. A stapled peptide, which binds a musculoaponeurotic fibrosarcoma (sMAF) protein and thereby inhibits its heterodimerization with nuclear factor erythroid 2-related factor 2 (NRF2), the stapled peptide comprising a peptide chain comprising at least two modified amino acids joined by at least one internal staple.

2. The stapled peptide of claim 1, wherein the peptide chain is about 15 amino acids to about 20 amino acids in length.

3. The stapled peptide of claim 1, wherein the peptide chain comprises a cell-penetrating peptide.

4. (canceled)

5. (canceled)

6. The stapled peptide of claim 1, which binds MafG with a kD of about 335 to about 340 nM.

7. (canceled)

8. The stapled peptide of claim 1, comprising SEQ ID NO: 6 or a functional variant thereof, wherein each X of SEQ ID NO: 6 is a modified amino acid, SEO ID NO: 7 or a functional variant thereof, wherein each X of SEQ ID NO: 7 is a modified amino acid, or SEQ ID NO: 8 or a functional variant thereof, wherein each X is a modified amino acid.

9. (canceled)

10. (canceled)

11. The stapled peptide of claim 8, wherein each X is or comprises (S)-2-amino-2-methyl-hept-6-enoic acid.

12. (canceled)

13. (canceled)

14. The stapled peptide of claim 1, wherein the at least one internal staple is a hydrocarbon tether or is pegylated.

15. (canceled)

16. (canceled)

17. The stapled peptide of claim 1, wherein the at least one internal staple comprises an olefin tether or a macrocyclic tether.

18. (canceled)

19. The stapled peptide of claim 1, wherein the at least one internal staple comprises about 6 to about 16 carbon atoms.

20. (canceled)

21. (canceled)

22. (canceled)

23. The stapled peptide of claim 1, wherein the at least one internal staple is at positions [i, i+4] of the peptide chain, wherein “i” represents the first staple position in the peptide chain.

24. (canceled)

25. The stapled peptide of claim 1, further comprising a tracer.

26. The stapled peptide of claim 25, wherein the tracer comprises a fluorescent molecule.

27. The stapled peptide of claim 1 modified by acetylation at a 5′-terminus thereof.

28. The stapled peptide of claim 1 comprising the amino acid sequence of R₁X₂R₃X₄R₅ (SEQ ID NO: 25), wherein:
R₁ is SEQ ID NO: 26, SEQ ID NO: 27, SEQ ID NO: 28, or has at least 80% sequence identity to SEQ ID NO: 26, SEQ ID NO: 27, or SEQ ID NO: 28;
R₃ is DLD, DEK, KEK, or has at least 66% sequence identity to DLD, DEK, or KEK;
R₅ is SEQ ID NO: 29, SEQ ID NO: 30, SEQ ID NO: 31, or is a functional variant of SEQ ID NO: 29, SEQ ID NO: 30, or SEQ ID NO: 31; and
X₂ and X₄ are modified amino acids joined by a first internal staple.

29. The stapled peptide of claim 28 comprising an amino acid sequence selected from the group consisting of SEQ ID NO: 6, SEQ ID NO: 7, and SEQ ID NO: 8.

30. The stapled peptide of claim 28, wherein the first internal staple comprises a hydrocarbon tether, an olefin tether, or a macrocyclic tether.

31. (canceled)

32. (canceled)

33. The stapled peptide of claim 28, wherein each X₂ and X₄ is or comprises (S)-2-amino-2-methyl-hept-6-enoic acid.

34. The stapled peptide of claim 28 comprising a helical conformation and, optionally, an α-helical conformation.

35. (canceled)

36. (canceled)

37. The stapled peptide of claim 28, wherein the at least one internal staple is at positions [i, i+4] of the peptide chain, wherein “i” represents the first staple position in the peptide chain.

38. The stapled peptide of claim 28, wherein SEQ ID NO: 25 is linked to or further comprises a cell-penetrating peptide.

39. A pharmaceutical composition comprising the stapled peptide of claim 1 and a pharmaceutically acceptable carrier and/or diluent.

40. A method of sensitizing a cancer to a cancer therapy, which method comprises contacting a cancer cell with:
a cancer therapy-sensitizing amount of a stapled peptide of claim 1 or a pharmaceutical composition comprising same and a pharmaceutically acceptable carrier and/or diluent;
whereupon the cancer cell is sensitized to the cancer therapy.

41. The method of claim 40, wherein the cancer is nuclear factor erythroid 2-related factor 2 (Nrf2)-addicted.

42. The method of claim 40, wherein the cancer therapy comprises the administration of a chemotherapy agent.

43. The method of claim 40, wherein the cancer therapy comprises administration of cisplatin.

44. The method of claim 40, wherein the cancer is cancer of the lung, breast or pancreas.

45. (canceled)

46. The method of claim 40, wherein the cancer therapy comprises an immune checkpoint inhibitor, an immune modulator, or a combination of both of the foregoing.

47. The method of claim 40, further comprising subjecting the cancer cell to radiation therapy, surgery, thermos-ablation, focused ultrasound therapy, cryotherapy, or any combination of the foregoing.

48. (canceled)

49. (canceled)

50. (canceled)

51. (canceled)

52. (canceled)

53. (canceled)
